(19) United States
(12) Patent Application Publication
(10) Pub. No.: US 2004/0039168 A1
(43) Pub. Date: Feb. 26, 2004

(54) Method for manufacture of nanostructures using staged-assembly

(76) Inventor: Edward B. Goldberg, Newton, MA (US)

Correspondence Address: OPPEDAHL AND LARSON LLP, P O BOX 5068, DILLON, CO, 80435-5068, US

(21) Appl. No.: 10/371,073

(22) Filed: Feb. 21, 2003

Related U.S. Application Data
(63) Continuation-in-part of application No. 10/136,225, filed on Apr. 29, 2002.
Application No. 10/136,225 is a division of application No. 09/226,949, filed on Jan. 8, 1999, now Pat. No. 6,197,139.
Application No. 09/226,949 is a division of application No. 08/542,003, filed on Oct. 12, 1995, now Pat. No. 5,864,013.
Application No. 08/542,003 is a continuation-in-part of application No. 08/322,760, filed on Oct. 13, 1994, now Pat. No. 5,877,279.

(51) Int. Cl. C07K 14/005; C12P 21/06

(52) U.S. Cl. 530/350; 435/68.1

(57) ABSTRACT

Nanostructures formed from a plurality of structural units in which the positions of the structural units relative to each other are established in a defined geometry, are formed by sequentially adding structural units to a growing structure to build up the nanostructure. The nanostructure includes two or more species of protein structural units, and each structural unit is added to the growing structure in a separate structural unit-addition step. Structural units not incorporated in the growing structure is removed at the end of each structural unit-addition step. Each species of structural unit has the ability to assemble non-covalently with the growing nanostructure to which it is added but cannot self-assemble with other structural units of the same species.

This application is a continuation-in-part of U.S. patent application Ser. No. 10/136,225, filed Apr. 29, 2002, which is a division of U.S. patent application Ser. No. 09/226,949, now U.S. Pat. No. 6,437,112, which is a division of U.S. patent application Ser. No. 08/542,003, filed Oct. 12, 1995, now U.S. Pat. No. 5,864,013, which is a continuation-in-part of U.S. patent application Ser. No. 08/322,760 filed Oct. 13, 1994, now U.S. Pat. No. 5,877,279, all of which are incorporated by reference herein in their entirety.

This invention was made with Government support under Grant No. MCB 9308834 awarded by the National Science Foundation. The Government has certain rights in this invention.

### FIELD OF THE INVENTION

The present invention pertains to the manufacture of nanostructures, i.e., nanometer-sized peptide structures useful in the construction of microscopic and macroscopic structures. In particular, the present invention pertains to a staged assembly process which can be used to manufacture such nanostructures.

### BACKGROUND TO THE INVENTION

While the strength of most metallic and ceramic based materials derives from the theoretical bonding strengths between their component molecules and crystallite surfaces, it is significantly limited by flaws in their crystal or glass-like structures. These flaws are usually inherent in the raw materials themselves or developed during fabrication and are often expanded due to exposure to environmental stresses.

The emerging field of nanotechnology has made the limitations of traditional materials more critical. The ability to design and produce very small structures (i.e., of nanometer dimensions) that can serve complex functions depends upon the use of appropriate materials that can be manipulated in predictable and reproducible ways, and that have the properties required for each novel application.

Biological systems serve as a paradigm for sophisticated nanostructures. Living cells fabricate proteins and combine them into structures that are perfectly formed and can resist damage in their normal environment. In some cases, intricate structures are created by a process of self-assembly, the instructions for which are built into, the component polypeptides. Finally, proteins are subject to proofreading processes that insure a high degree of quality control.

Therefore, there is a need in the art for methods and compositions that exploit these unique features of proteins to form constituents of synthetic nanostructures. The need is to design materials whose properties can be tailored to suit the particular requirements of nanometer-scale technology. Moreover, since the subunits of most macrostructural materials, ceramics, metals, fibers, etc., are based on the bonding of nanostructural subunits, the fabrication of appropriate subunits without flaws and of exact dimensions and uniformity should improve the strength and consistency of the macrostructures because the surfaces are more regular and can interact more closely over an extended area than larger, more heterogeneous material.

### SUMMARY OF THE INVENTION

The present invention provides a method for making a nanostructure comprising sequentially adding individual building blocks or structural units to a growing structure to build up the nanostructure. In the method of the invention, each structural unit is added to the growing structure in a separate structural unit-addition step. Between structural unit-addition steps, structural units that have not been incorporated in the growing structure are removed. Each structural unit in the nanostructure is chosen such that it has the ability to bind to assemble with the growing nanostructure to which it is added but cannot self-assemble with other components of the same species. The final nanostructure comprises two or more species of protein structural units.

In one aspect of the present invention, some or all of the protein structural units used as the building blocks for nanostructures comprise native or modified tail fiber proteins of T-even bacteriophage such as bacteriophage T4. For example, protein structural units derived from T4 gp34, 36, and 37 proteins, which can be modified in various ways to form novel rod structures with different properties, can be used. The structural units may also be fusion proteins, for example fusion proteins that contain sequences from two or more different tail fiber proteins, or from combinations of tail fiber proteins with others proteins or peptides.

### BRIEF DESCRIPTION OF THE DRAWINGS

FIGS. 1A and 1B show a schematic representation of the T4 bacteriophage particle (FIG. 1A), and a schematic representation of the T4 bacteriophage tail fiber (FIG. 1B).

FIG. 2 shows a schematic representation of a unit rod.

FIGS. 3A-3D show schematic representations of: a one-dimensional multi-unit rod joined along the x axis (FIG. 3A); closed simple sheets (FIG. 3B); closed brickwork sheets (FIG. 3C); and open brickwork sheets (FIG. 3D).

FIG. 4 shows a schematic representation of two units used to construct porous and solid sheets (top and bottom), which, when alternatively layered, produce a multi-tiered set of cages as shown.

FIG. 5 shows a schematic representation of an angled structure having an angle of 120°.

FIGS. 6A-6D show the DNA sequence (SEQ ID NO: 1) of genes 34, 35, 36, and 37 of bacteriophage T4.

FIGS. 7A-Q show the amino acid sequences (shown in single-letter codes) of the gene products of genes 34 (SEQ ID NO: 2, ORFX SEQ ID NO: 3), 35 (SEQ ID NO: 4), 36 (SEQ ID NO: 5), and 37 (SEQ ID NO: 6) of bacteriophage T4. The amino acid sequences (bottom line of each pair) are aligned with the nucleotide sequences (top line of each pair.) It is noted that the deduced protein sequence of gene 35 (from NCBI database) is not believed to be accurate.

FIGS. 8A-8B show a schematic representation of: the formation of a P37 dimer initiator from a molecule that self-assembles into a dimer (FIG. 8A); and the formation of a P37 trimer initiator from a molecule that self-assembles into a trimer (FIG. 8B).

FIG. 9 shows a schematic representation of the formation of the polymer (P37-36)n with an initiator that is a self-assembling multimer.

FIGS. 10A-D show inactivation of phage by a monoclonal antibody. (A) Treatment of phage with mAb and secondary anti-serum. Each phage type was treated with 1 μg of mAb as described in Materials and Methods. (B) Time course of mAb treatment. SΔ1ras2 phage were treated with 3 μg of mAb for the indicated time before a 30 min incubation with secondary anti-serum. (C) Dose-response study of SΔ1ras2 phage with varying amounts of mAb. (D) Effect of treating mAb with free epitope before inactivation of SΔ1ras2.

### DETAILED DESCRIPTION OF THE INVENTION

All patents, patent applications and literature references cited in the specification are hereby incorporated by reference in their entirety. In the case of inconsistencies, the present disclosure, including, definitions, will prevail.

Although the invention is described in terms of bacteriophage T4 tail fiber proteins, it will be understood, that the invention is also applicable to tail fiber proteins of other T-even-like phage, e.g., of the T4 family (e.g., T4, TuIa, TuIb), and T2 family (T2, T6, K3, O×2, M1, etc.)

Definitions

“Nanostructures” are defined herein as structures of different sizes and shapes that are assembled from nanometer-sized structural units in which the positions of the structural units relative to each other are established in a defined geometry. The nanostructure may have functional substitutents attached to it.

“Chimers” are defined herein as chimeric proteins in which at least the amino- and carboxy-terminal regions are derived from different original polypeptides, whether the original polypeptides are naturally occurring or have been modified by mutagenesis.

“Homomultimers” are defined herein as assemblies of two or three substantially identical protein subunits that form a defined three-dimensional structure.

The designation “gp” denotes a monomeric polypeptide, while the designation “P” denotes homooligomers. P34, P36, and P37 are presumably homodimers or homotrimers.

An isolated polypeptide that “consists essentially of” a specified amino acid sequence is defined herein as a polypeptide having the specified sequence or a polypeptide that contains conservative substitutions within that sequence. Conservative substitutions, as those of ordinary skill in the art would understand, are ones in which an acidic residue is replaced by an acidic residue, a basic residue by a basic residue, or a hydrophobic residue by a hydrophobic residue. Also encompassed is a polypeptide that lacks one or more amino acids at either the amino terminus or carboxy terminus, up to a total of five at either terminus, when the absence of the particular residues has no discernable effect on the structure or the function of the polypeptide in practicing the present invention.

In this application, the term “protein structural units” is used generically to referred to structural units containing peptides, polypeptides and proteins that comprise a plurality of amino acids, and is not intended to imply any minimum number of amino acids.

In this application the terms “removing” or “removal” of unbound structural units is accomplished when they are rendered unable to participate in further reactions with the growing nanostructure, whether or not they are physically removed.

The present invention may make use of a new class of T-even tail fiber protein building blocks whose dimensions are measured in nanometers, which are useful in the construction of microscopic and macroscopic structures. Without wishing to be bound by theory, it is now believed that the basic unit of T-even tail fiber proteins is a homotrimer composed of three identical protein subunits, although previously it was though that a dimeric structure might be correct. The term “homomultimer” will be used to encompass either this homotrimer structure of the previously-favored homodimer possibility. The homomultimers interact as β-sheets to form long, stiff, and stable rod-shaped structural units that can assemble with other rods using coupling devices that can be attached genetically or in vitro. The ends of one rod may attach to different ends of other rods or similar rods. Variations in the length of the rods, in the angles of attachment, and in their flexibility characteristics permit differently-shaped structures to be assembled. In this manner the units can be assembled into predetermined larger structures of one, two or three dimensions. In the method of the present invention, the assembly is staged to form structures of precise dimensions and uniform length due to the flawless biological manufacture of the components. The rods can also be modified by genetic and chemical modifications to form predetermined specific attachment sites for other chemical entities, allowing the formation of complex structures.

An important aspect of the present invention is that the protein structural units can be designed so that they comprise rods of different lengths which can be used to make nanostructures with different defined geometries, and can be further modified to include features that alter their surface properties in predetermined ways and/or influence their ability to join with other identical or different units. Furthermore, the self-assembly capabilities can be expanded by producing chimeric proteins that combine the properties of two different members of this class. This design feature is achieved by manipulating the structure of the genes encoding these proteins.

As detailed below, the methods of the present invention take advantage of the properties of the natural proteins, i.e., the resulting structures are stiff, strong, stable in aqueous media, heat resistant, protease resistant, and can be rendered biodegradable. A large quantity of units can be fabricated easily in microorganisms. Furthermore, for ease of automation, large quantities of parts and subassemblies can be stored and used as needed.

The sequences of a specific class of the protein structural units are based on the components of the tail fiber of the T4 bacteriophage of E. coli. It will be understood that the principles and techniques can be applied to the tail fibers of other T-even phages, or other related bacteriophages that have similar tail and/or fiber structures.

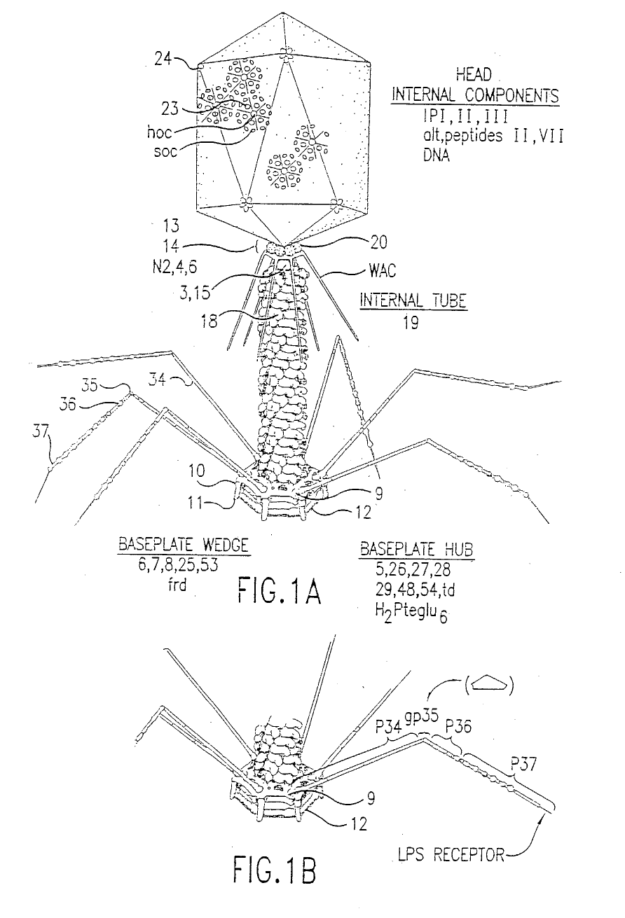

The structure of the T4 bacteriophage tail fiber (illustrated in FIGS. 1A-1B) can be represented schematically as follows (N=amino terminus, C=carboxy terminus): N[P34]C—N[gp35]C—N[P36]C—N[P37]C. P34, P36, and P37 are all stiff, rod-shaped protein homomultimers in which identical β sheets, oriented in the same direction, are fused by hydrophobic interactions between the sheets juxtaposed with a rotational axis of symmetry through the long axis of the rod. gp35, by contrast, is a monomeric polypeptide that attaches specifically to the N-terminus of P36 and then to the C-terminus of P34 and forms an angle joint between two rods. During T4 infection of E. coli, gp37 monomers multimerize to form a P37 homomultimer; the process of multimerization is believed to initiate near the C-terminus of P37 and to require two E. coli chaperon proteins. (A variant gp37 with a temperature sensitive mutation near the C-terminus used in the present invention requires only one chaperon, gp57, for multimerization.) Once assembled, the N-terminus of P37 initiates the multimerization of gp36 monomers to a P36 rod. The joint between the C-terminus of P36 and the N-terminus of P37 is tight and stiff but noncovalent. The N-terminus of P36 then attaches to a gp35 monomer; this interaction stabilizes P36 and forms the elbow of the tail fiber. Finally, gp35 attaches to the C-terminus of P34 (which uses gp57 for multimerization). Thus, self assembly of the tail fiber is regulated by a predetermined order of interaction of specific subunits whereby structural maturation caused by formation of the first subassembly permits interaction with new (previously disallowed) subunits. This results in the production of a structure of exact specifications from a random mixture of the components.

In accordance with the present invention, the genes encoding these proteins may be modified so as to make rods of different lengths with different combinations of ends. The properties of the native proteins are particularly advantageous in this regard. The core sections of the protein can be shortened or lengthened by genetic manipulations e.g., by splicing DNA regions encoding β-bends, on the same edge of the sheet, to form new bends that exclude intervening peptides, or by inserting segments of peptide in an analogous manner by splicing at bend angles. This property allows amino acid side chains extending above and below the surface of the protein to be modified by genetic substitution or chemical coupling. Importantly, all of the above modifications are achieved without compromising the structural integrity of the rod. It will be understood by one skilled in the art that these properties allow a great deal of flexibility in designing units that can assemble into a broad variety of structures, some of which are detailed below.

Structural Units

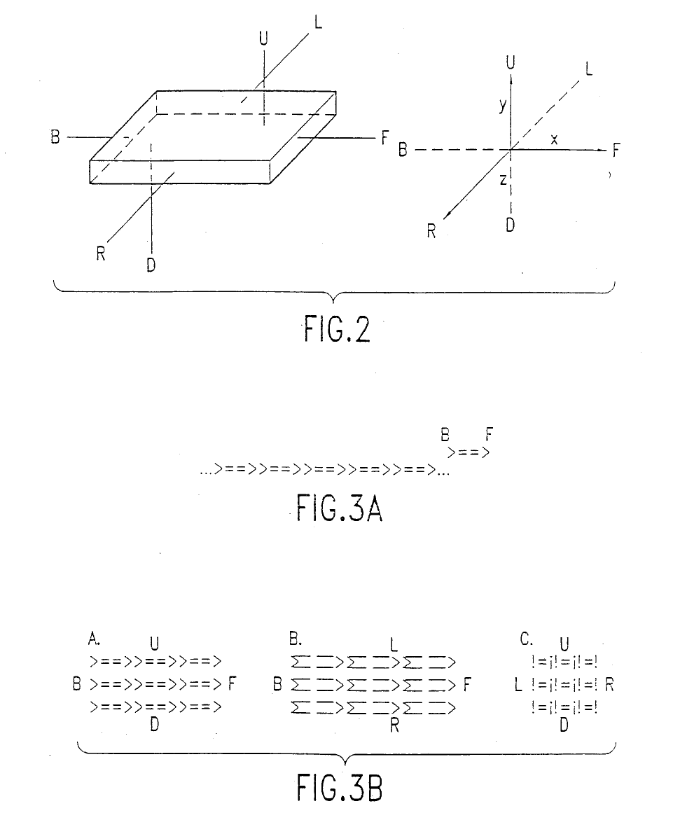

Rod-like protein sub-units are used in the method of the invention like wooden 2×4 studs or steel beams for construction of the nanostructure. In this case, the surfaces are exactly reproducible at the molecular level and thereby fitted for specific attachments to similar or different units rods at fixed-joining sites. The surfaces are also modified to be more or less hydrophilic, including positively or negatively charged groups, and have protrusions built in for specific binding to other units or to an intermediate joint with two receptor sites. The surfaces of the rod and a schematic of the unit rod are illustrated in FIG. 2. The three dimensions of the rod are defined as: x, for the back (B) to front (F) dimension; y, for the down (D) to up (U) dimension; and z, for the left (L) to right (R) dimension. In the now accepted trimeric formation, the aspect ratio of the structure may be less than that shown in FIG. 2.

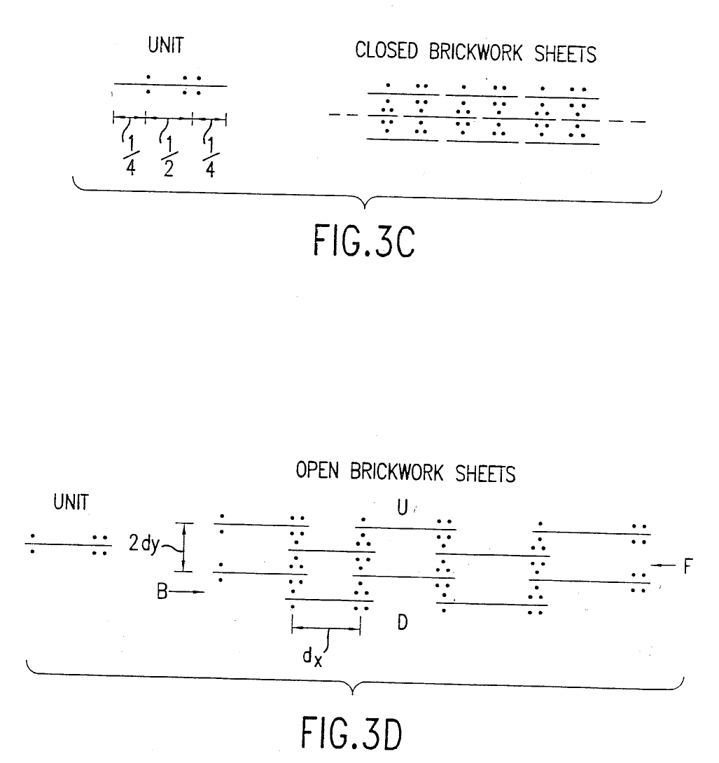

One dimensional multi-unit rods can be most readily assembled from single unit rods joined along the x axis (FIG. 3A) but regular joining of subunits in either of the other two dimensions will also form a long structure, but with different cross sections than in the x dimension.

Two dimensional constructs are sheets formed by interaction of rods along any two axes. 1) Closed simple sheets are formed from surfaces which overlap exactly, along any two axes (FIG. 3B). 2) Closed brickwork sheets are formed from interaction between units that have exactly overlapping surfaces in one dimension and a special type of overlap in the other (FIG. 3C). In this case there must be two different sets of complementary joints spaced with exactly ½ unit distance between them. If they are centered (i.e., each set ¼ from the end) then each joint will be in the center of the units above and below. If they are offset, then the joint will be offset as well. In this construction, the complementary interacting sites are schematized by • and • •. If the interacting sites are each symmetric, the alternating rows can interact with the rods in either direction. If they are not symmetric, and can only interact with interacting rows facing in the same or opposite direction, the sheet will made of unidirectional rods or layers of rods in alternating directions. 3) Open brickwork sheets (or nets) result when the units are separated by more than one-half unit (FIG. 3D). The dimensions of the openings (or pores) depend upon the distance (dx) separating the interacting sites and the distance (dy) by which these sites separate the surfaces.

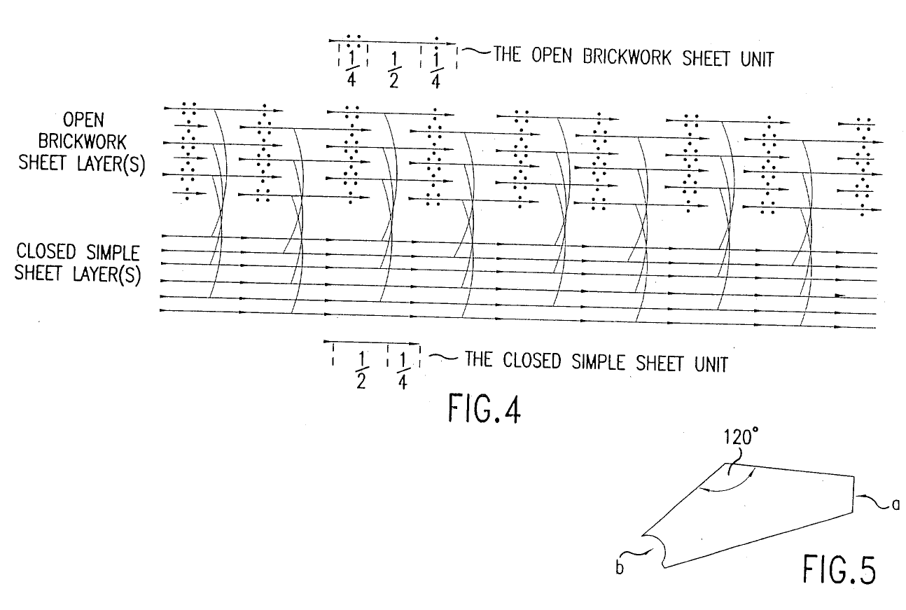

Three dimensional constructs require sterically compatible interactions between all three surfaces to form solids. 1) Closed solids can assemble from units that overlap exactly in all three dimensions (e.g., the exact overlapping of closed simple sheets). In an analogous manner, closed brickwork sheets can form closed solids by overlapping sheets exactly or displaced to bring the brickwork into the third dimension. This requires an appropriate set of joints on all three pairs of parallel faces of the unit. 2) Porous solids are made by joining open brickwork sheets in various ways. For example, if the units overlap exactly in the third dimension, a solid is formed with the array of holes of exact dimensions running perpendicular to the plane of the paper. If instead, a material is needed with closed spaces, with layers of width dz (i.e., in the U→D dimension), a simple closed sheet is layered on the open brickwork sheet to close the openings. If the overlap of the open brickwork sheet is e.g., ¼ unit, then a rod of length ¾ units is used to make the sheet. Joints are then needed in the z dimension. The two units used to polymerize these alternate layers, and the layers themselves, are schematized in FIG. 4.

All of the above structures are composed of simple linear rods. A second unit, the angle unit, expands the type and dimensionality of possible structures. The angle unit connects two rods at angles different from 180°, akin to an angle iron. The average angle and its degree of rigidity are built into this connector structure. For example, the structure shown in FIG. 5 has an angle of 120° and different specific joining sites at a and at b. The following are examples of structures that are formed utilizing angle joints:

1) Open brickwork sheets are expanded and strengthened in the direction normal to the rod direction by adding angles perpendicular to the sheet. In this case, a three dimensional network forms. Attachment of 90° angles to the ends of the rods makes an angle almost in the plane of the sheet, allowing new rods added to those angles (which must have some play out of the plane of the original sheet to attach in the first place) to form a new sheet, almost parallel, with an orientation normal to its upper or lower neighbor.

2) Hexagons are made from a mixture of rods and angle joints that form 120° angles. In this case, there are two exclusive sets of joints. Each set is made up of one of the two ends of the rod and one of the two complementary sites on the angle. This is a linear structure in the sense that the hexagon has a direction (either clockwise or counterclockwise). It can be made into a two dimensional open net (i.e., a two dimensional honeycomb) by joining the sides of the hexagons. It can form hexagonal tubes by joining the top of the hexagon below to the bottom face of the hexagon above. If the tubes also join by their sides, they will form an open three dimensional multiple hexagonal tube.

3) Helical hexagonal tubes are made analogously to hexagons but the sixth unit is not joined to the first to close the hexagon. Instead, the end is displaced from the plane of the hexagon and the seventh and further units are added to form a hexagonal tube which can be a spring if there is little or no adhesive force between the units of the helix, or a stiff rod if there is such a force to maintain the close proximity of apposing units.

It will be apparent to one skilled in the art that the compositions and methods of the present invention also encompass other polygonal structures such as octagons, as well as open solids such as tetrahedrons and icosahedrons formed from triangles and boxes formed from squares and rectangles. The range of structures is limited only by the types of angle units and the substituents that can be engineered on the different axes of the rod units. For example, other naturally occurring angles are found in the fibers of bacteriophage T7, which has a 90° angle (Steven et al., J. Mol. Biol. 200: 352-365, 1988).

Design and Production of Rod Proteins for Use as Structural Units

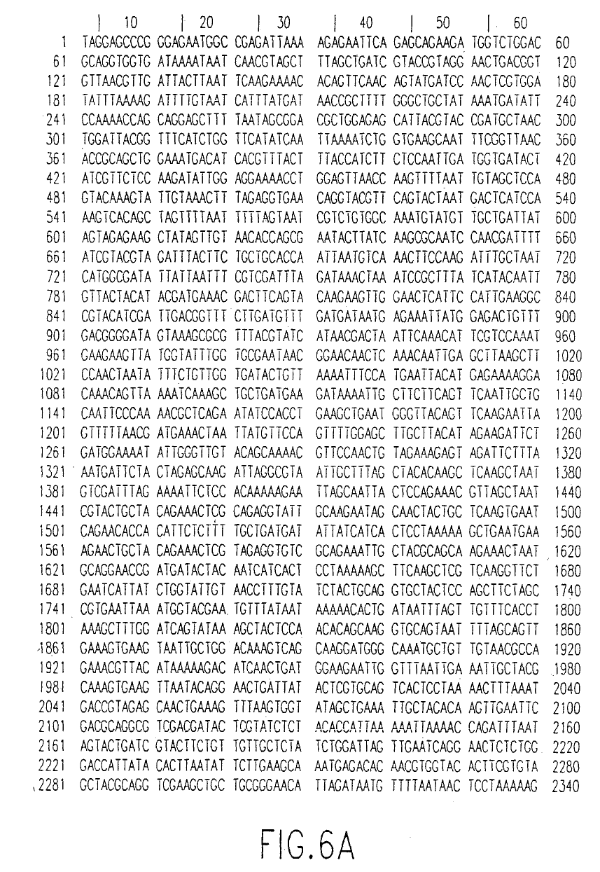

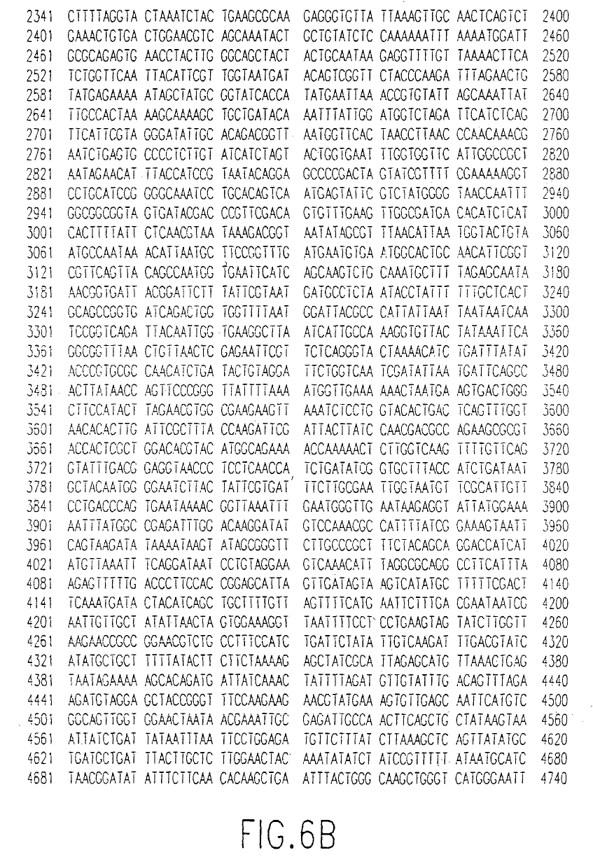

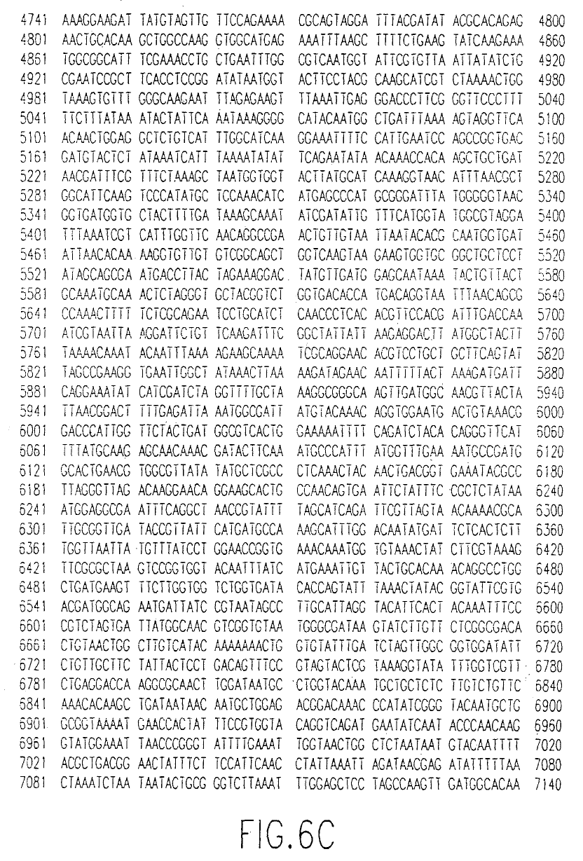

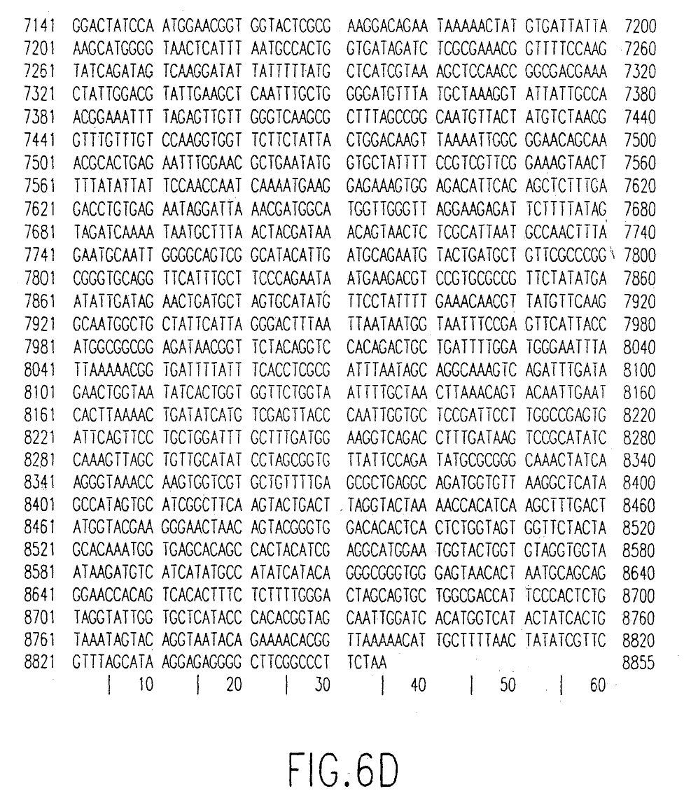

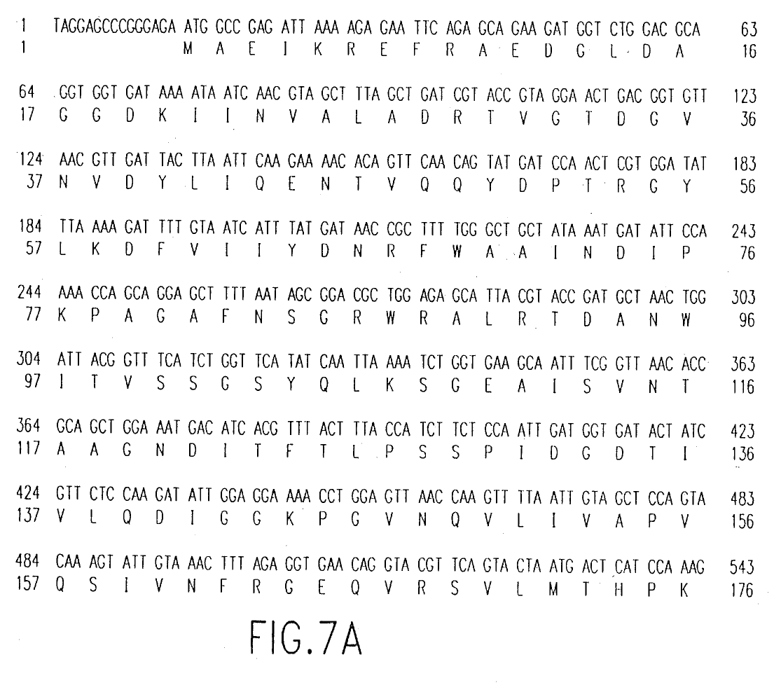

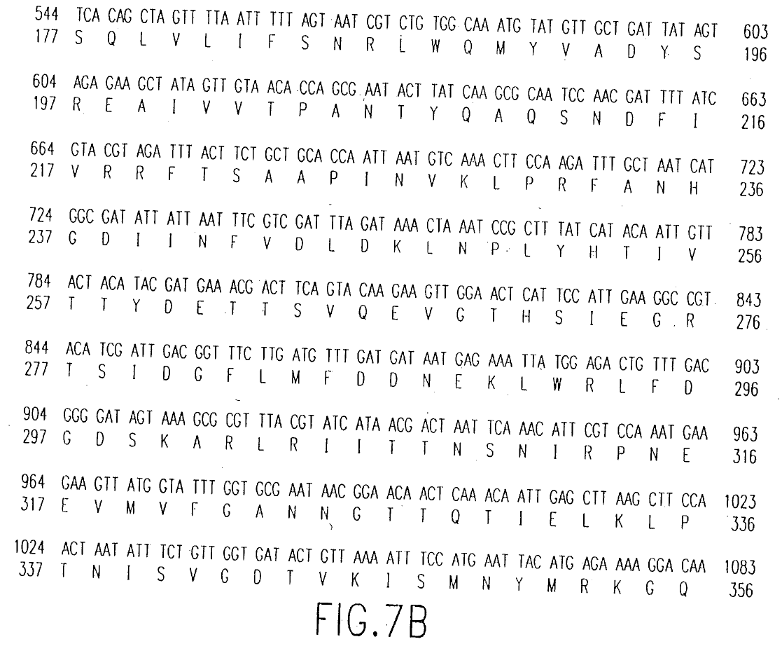

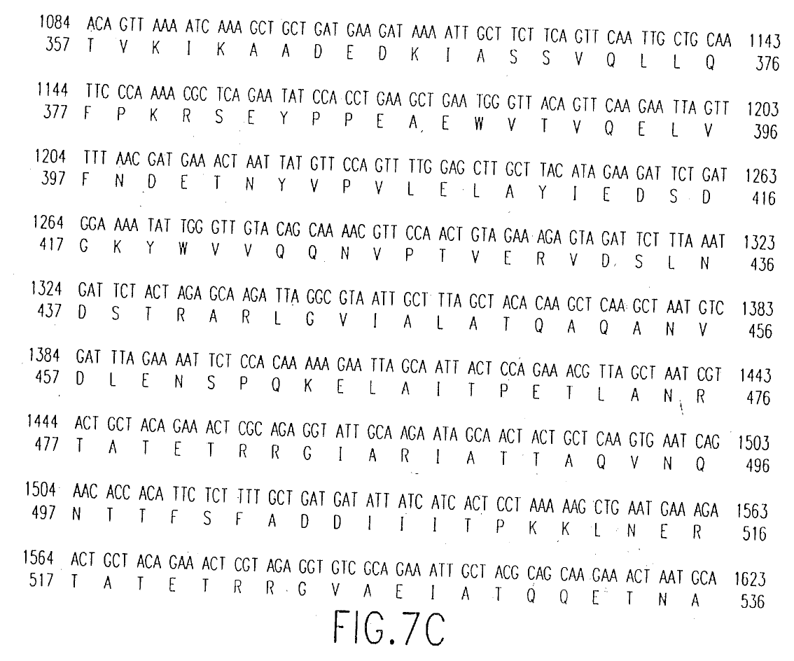

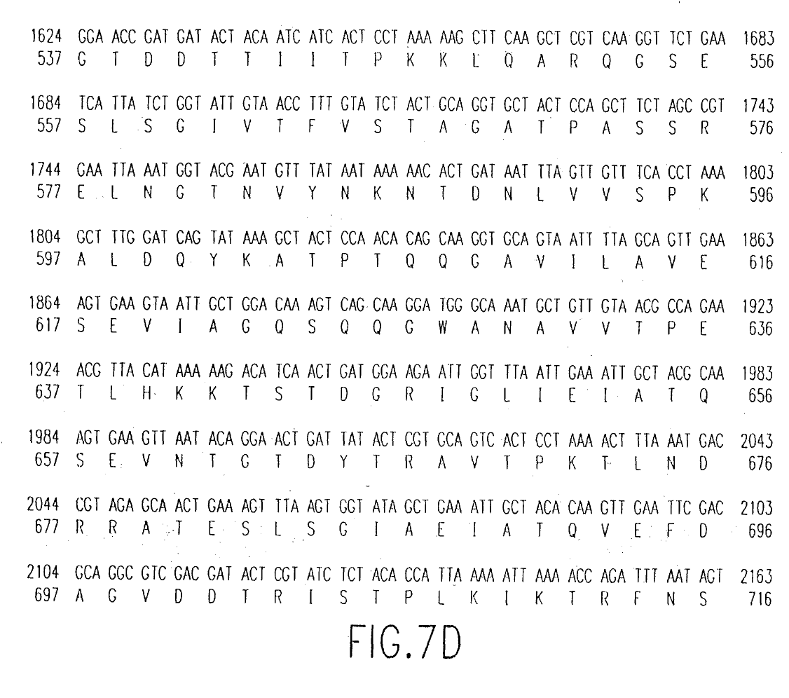

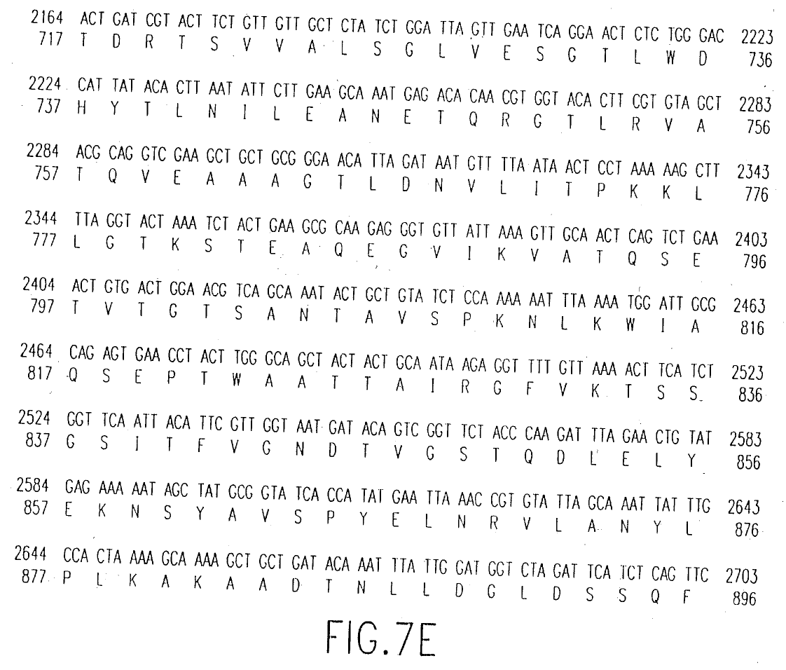

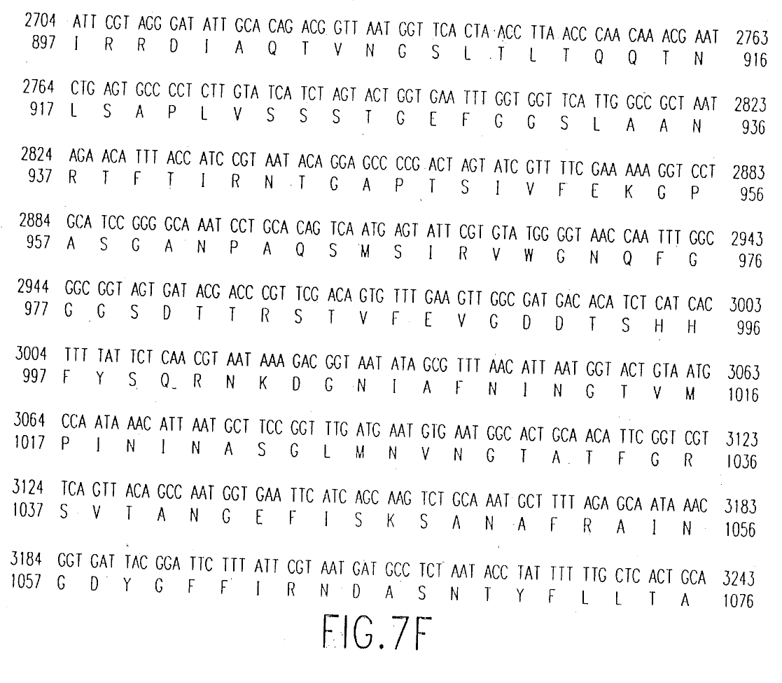

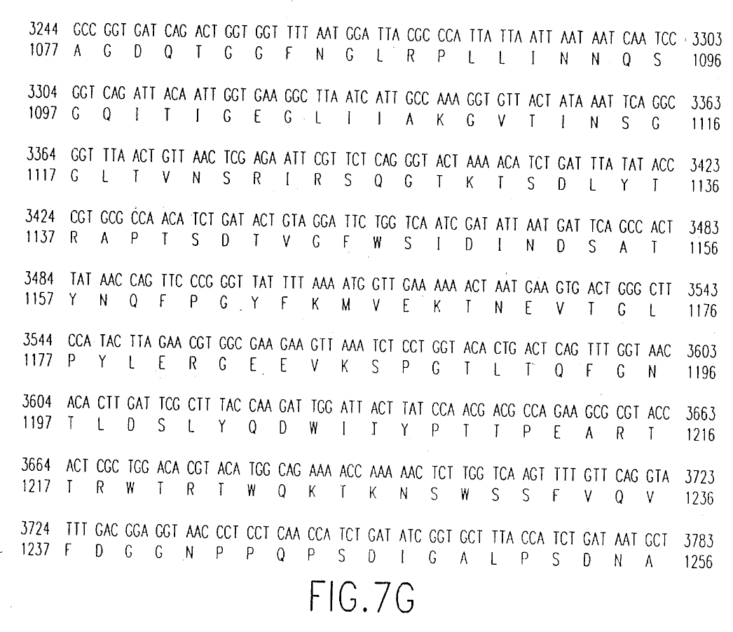

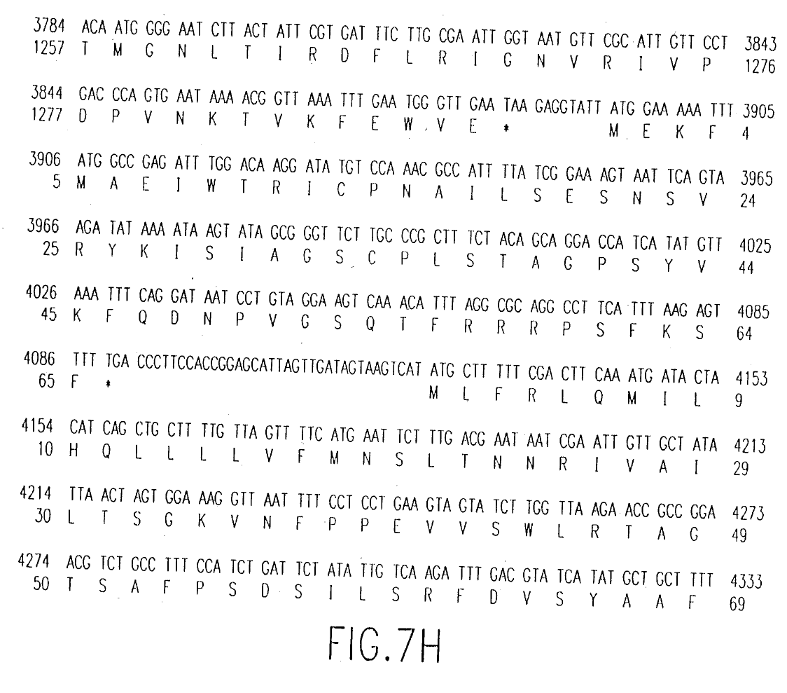

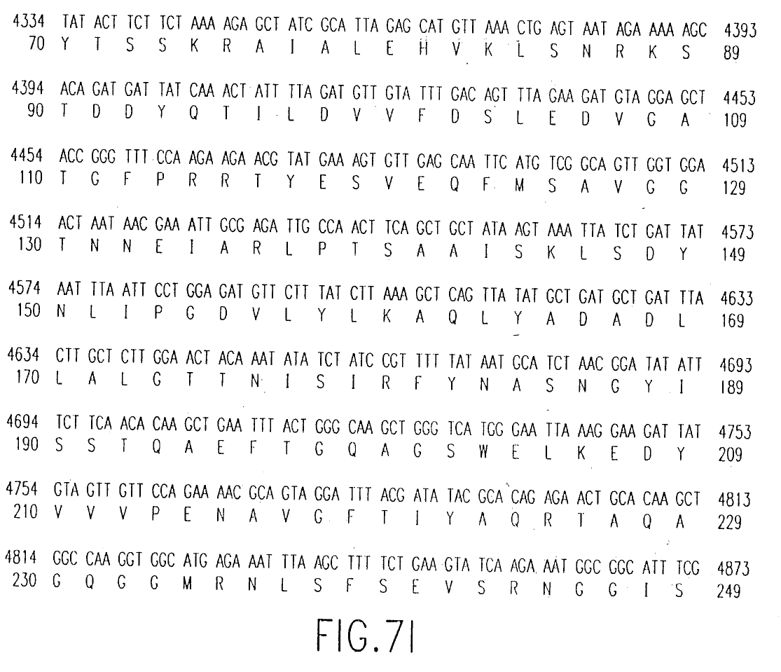

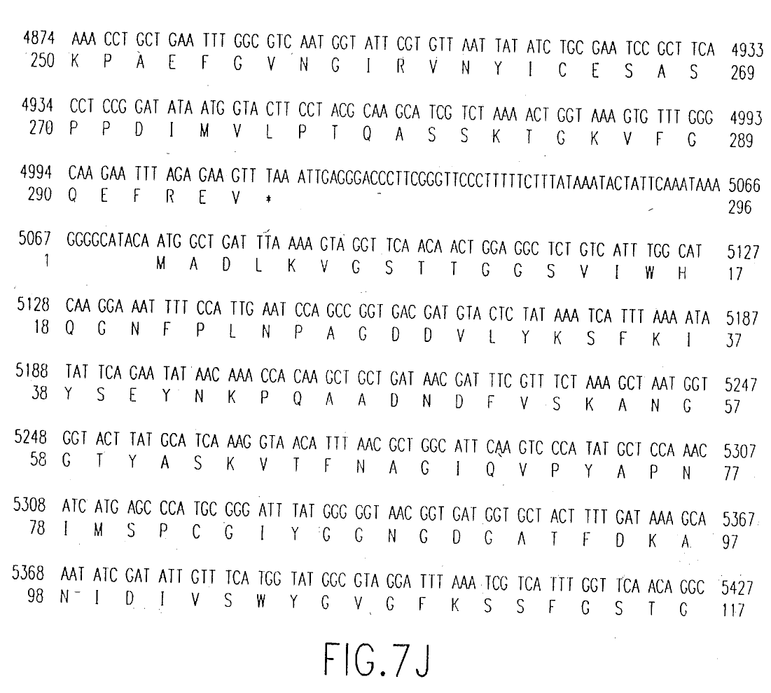

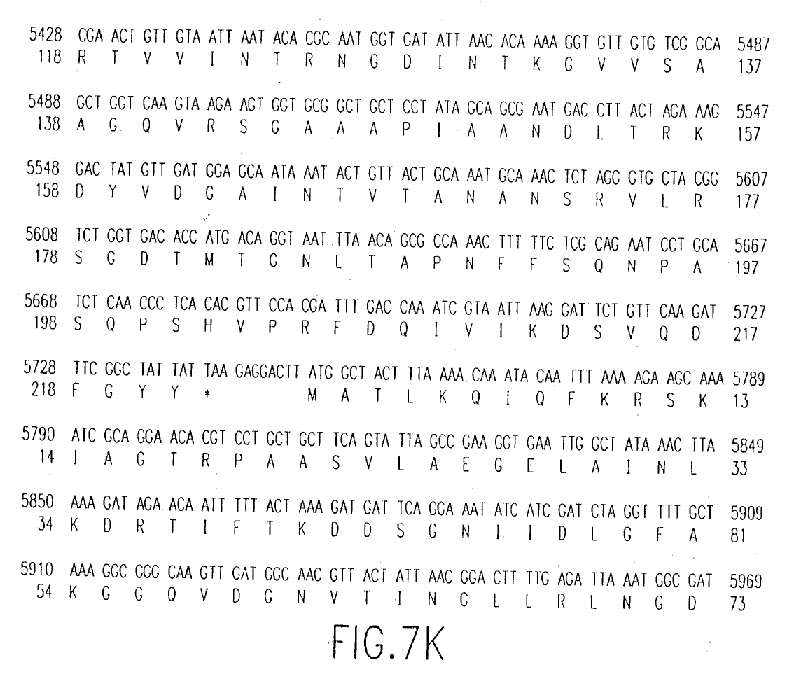

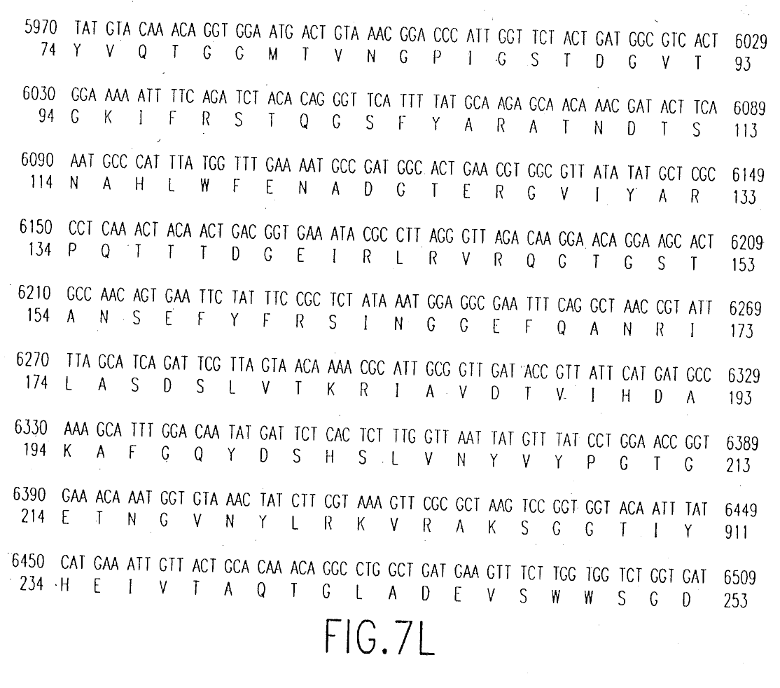

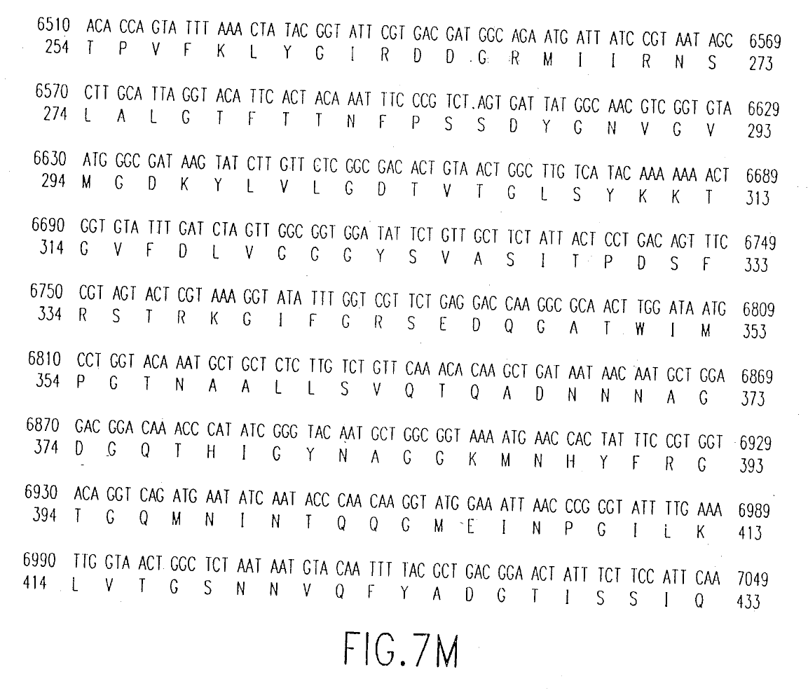

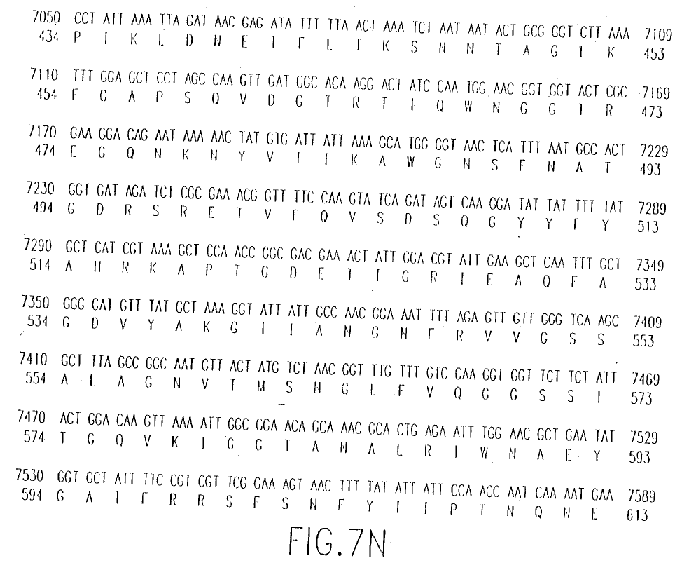

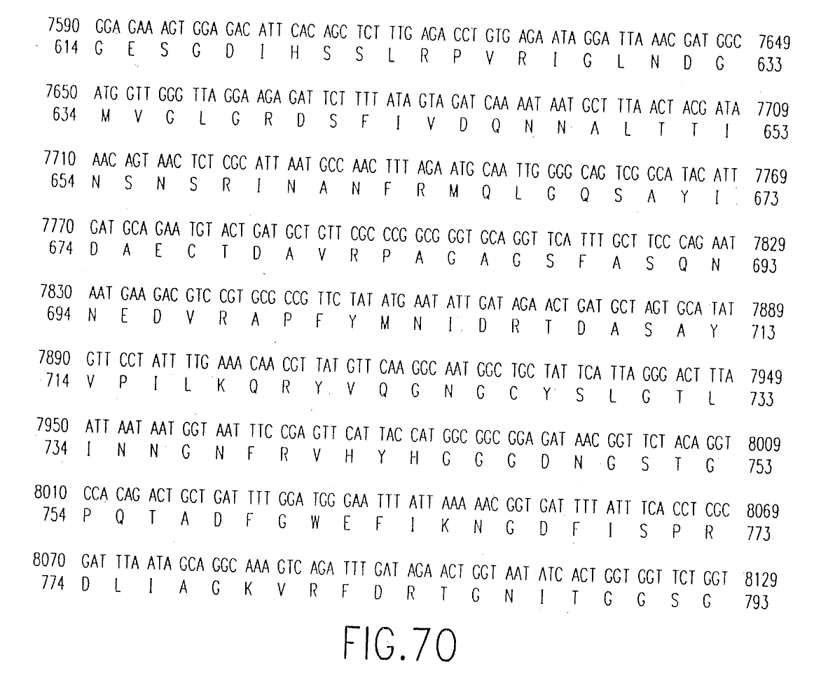

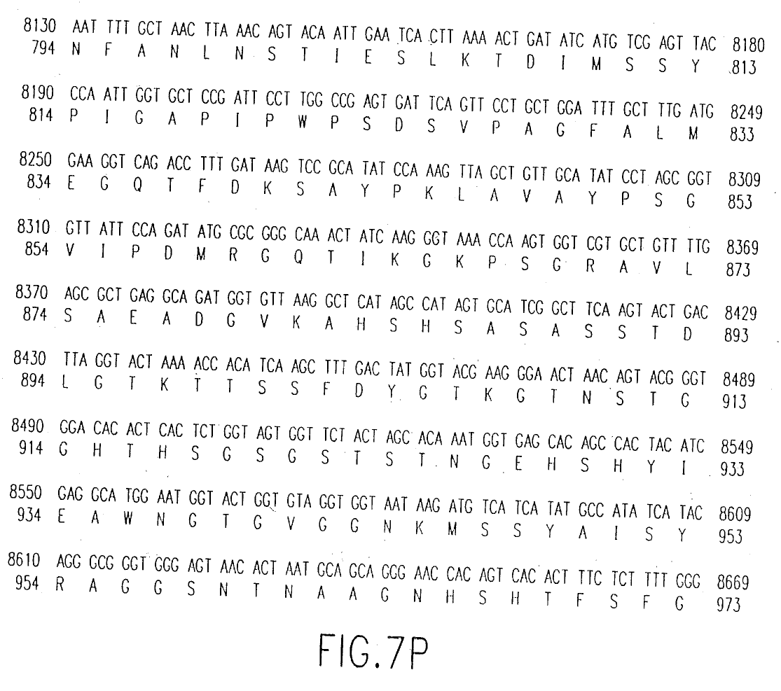

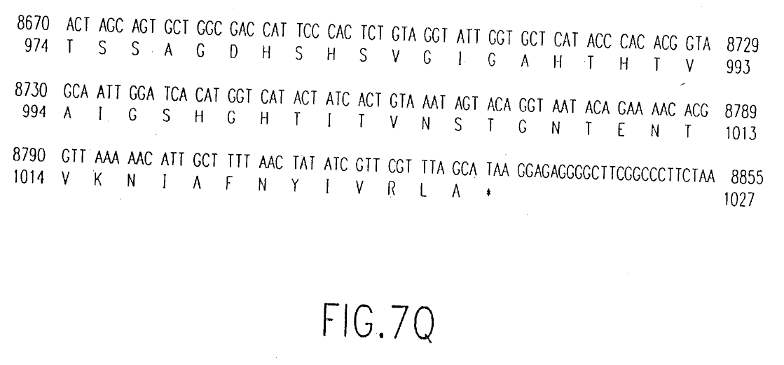

Protein structural units that can be used to construct the nanostructures in accordance with the method of the present invention can be based on the four polypeptides that comprise the tail fibers of bacteriophage T4, i.e., gp34, gp35, gp36 and gp37. The genes encoding these proteins have been cloned, and their DNA and protein sequences have been determined (for gene 36 and 37 see Oliver et al. J. Mol. Biol. 153: 545-568, 1981). The DNA and amino acid sequences of genes 34, 35, 36 and 37 are set forth in FIGS. 6A-D and 7A-D below.

Gp34, gp35, gp36, and gp37 are produced naturally following infection of E. coli cells by intact T4 phage particles. Following synthesis in the cytoplasm of the bacterial cell, the gp34, 36, and 37 monomers form homomultimers, which are competent for assembly into maturing phage particles. Thus, E. coli serves as an efficient and convenient factory for synthesis and multimerization of the protein subunits described herein below.

In practicing the present invention, the genes encoding the proteins of interest (native, modified, or recombined) are incorporated into DNA expression vectors that are well known in the art. These circular plasmids typically contain selectable marker genes (usually conferring antibiotic resistance to transformed bacteria), sequences that allow replication of the plasmid to high copy number in E. coli, and a multiple cloning site immediately downstream of an inducible promoter and ribosome binding site. Examples of commercially available vectors suitable for use in the present invention include the pET system (Novagen, Inc., Madison, Wis.) and Superlinker vectors pSE280 and pSE380 (Invitrogen, San Diego, Calif.).

The strategy is to 1) construct the gene of interest and clone it into the multiple cloning site; 2) transform E. coli cells with the recombinant plasmid; 3) induce the expression of the cloned gene; 4) test for synthesis of the protein product; and, finally, 5) test for the formation of functional homomultimers. In some cases, additional genes are also cloned into the same plasmid, when their function is required for multimerization of the protein of interest. For example, when wild-type or modified versions of gp37 are expressed, the bacterial chaperon gene 57 is also included; when wild-type or modified gp36 is expressed, the wild-type version or a modified version of the gp37 gene is included. The modified gp37 should have the capacity to multimerize and contain an N-terminus that can chaperon the multimerization of gp36. This method allows the formation of monomeric gene products and, in some cases, maturation of monomers to homomultimeric rods in the absence of other phage-induced proteins normally present in a T4-infected cell.

Steps 1-4 of the above-defined strategy are achieved by methods that are well known in the art of recombinant DNA technology and protein expression in bacteria. For example, in step 1, restriction enzyme cleavage at multiple sites, followed by ligation of fragments, is used to construct deletions in the internal rod segment of gp34, 36, and 37 (see Example 1 below). Alternatively, a single or multiple restriction enzyme cleavage, followed by exonuclease digestion (EXO-SIZE, New England Biolabs, Beverly, Mass.), is used to delete DNA sequences in one or both directions from the initial cleavage site; when combined with a subsequent ligation step, this procedure produces a nested set of deletions of increasing sizes. Similarly, standard methods are used to recombine DNA segments from two different tail fiber genes, to produce chimeric genes encoding fusion proteins (called “chimers” in this description). In general, this last method is used to provide alternate N- or C-termini and thus create novel combinations of ends that enable new patterns of joining of different rod segments. A representative of this type of chimer, the fusion of gp37-36, is described in Example 2. The preferred hosts for production of these proteins (Step 2) is E. coli strain BL21(DE3) and BL21(DE3/pLysS) (available commercially from Novagen, Madison, Wis.), although other compatible recA-strains, such as HMS174(DE3) and HMS174(DE3/pLysS) can be used. Transformation with the recombinant plasmid (Step 2) is accomplished by standard methods (Sambrook, J., Molecular Cloning, Cold Spring Harbor Laboratories, Cold Spring Harbor, N.Y.; this is also the source for standard recombinant DNA methods used in this invention.) Transformed bacteria are selected by virtue of their resistance to antibiotics e.g., ampicillin or kanamycin. The method by which expression of the cloned tail fiber genes is induced (Step 3) depends upon the particular promoter used. A preferred promoter is plac (with a laci^{q} on the vector to reduce background expression), which can be regulated by the addition of isopropylthiogalactoside (IPTG). A second preferred promoter is pT7φ10, which is specific to T7 RNA polymerase and is not recognized by E. coli RNA polymerase. T7 RNA polymerase, which is resistant to rifamycin, is encoded on the defective lambda DE lysogen in the E. coli BL21 chromosome. T7 polymerase in BL21(DE3) is super-repressed by the laci^{q} gene in the plasmid and is induced and regulated by IPTG.

Typically, a culture of transformed bacteria is incubated with the inducer for a period of hours, during which the synthesis of the protein of interest is monitored. In the present instance, extracts of the bacterial cells are prepared, and the T4 tail fiber proteins are detected, for example, by SDS-polyacrylamide gel electrophoresis.

Once the modified protein is detected in bacterial extracts, it is necessary to ascertain whether or not it forms appropriate homomultimers (Step 4). This is accomplished initially by testing whether the protein is recognized by an antiserum specific to the mature multimerized form of the protein.

Tail fiber-specific antisera are prepared as described (Edgar, R. S. and Lielausis, I., Genetics 52: 1187, 1965; Ward et al, J. Mol. Biol. 54:15, 1970). Briefly, whole T4 phage are used as an immunogen; optionally, the resulting antiserum is then adsorbed with tail-less phage particles, thus removing all antibodies except those directed against the tail fiber proteins. In a subsequent step, different aliquots of the antiserum are adsorbed individually with extracts that each lack a particular tail fiber protein. For example, if an extract containing only tail fiber components P34, gp35, and gp36 (derived from a cell infected with a mutant T4 lacking a functional gp37 gene) is used for absorption, the resulting antiserum will recognize only mature P37 and multimerized P36-P37. A similar approach may be used to prepare individual antisera that recognize only mature (i.e., homomultimerized) P34 and P36 by adsorbing with extracts containing distal half tail fibers or P34, gp35 and P37, respectively. An alternative is to raise antibody against purified tail fiber halves, e.g., P34 and gp35-P36-P37. Anti gp35-P36-P37 can then be adsorbed with P36-P37 to produce anti-gp35, and anti-P36 can be produced by adsorption with P37 and gp35. Anti-P37, anti-gp35, and anti-P34 can also be produced directly by using purified P37, gp35, and P34 as immunogens. Another approach is to raise specific monoclonal antibodies against the different tail fiber components or segments thereof.

Specific antibodies to subunits or tail parts are used in any of the following ways to detect appropriately homomultimerized tail fiber proteins: 1) Bacterial colonies are screened for those expressing mature tail fiber proteins by directly transferring the colonies, or, alternatively, samples of lysed or unlysed cultures, to nitrocellulose filters, lysing the bacterial cells on the filter if necessary, and incubating with specific antibodies. Formation of immune complexes is then detected by methods widely used in the art (e.g., secondary antibody conjugated to a chromogenic enzyme or radiolabelled Staphylococcal Protein A.). This method is particularly useful to screen large numbers of colonies e.g., those produced by EXO-SIZE deletion as described above. 2) Bacterial cells expressing the protein of interest are first metabolically labelled with ³⁵S-methionine, followed by preparation of extracts and incubation with the antiserum. The immune complexes are then recovered by incubation with immobilized Protein A followed by centrifugation, after which they may be resolved by SDS-polyacrylamide gel electrophoresis.

An alternative competitive assay for testing whether internally deleted tail fiber proteins that do not permit phage infection nonetheless retain the ability to multimerize and associate with their appropriate partners utilizes an in vitro complementation system. 1) A bacterial extract containing the modified protein of interest, as described above, is mixed with a second extract prepared from cells infected with a T4 phage that is mutant in the gene of interest. 2) After several hours of incubation, a third extract is added that contains the wild-type version of the protein being tested, and incubation is continued for several additional hours. 3) Finally, the extract is titered for infectious phage particles by infecting E. coli and quantifying the phage plaques that result. A modified tail fiber protein that is correctly multimerized and able to join with its partners is incorporated into tail fibers in a non-functional manner in Step 1, thereby preventing the incorporation of the wild-type version of the protein in Step 2; the result is a reduction in the titer of the resulting phage sample. By contrast, if the modified protein is unable to multimerize and thus form proper N- and/or C-termini, it will not be incorporated into phage particles in Step 1, and thus will not compete with assembly of intact phage particles in Step 2; the phage titer should thus be equivalent to that observed when no modified protein is added in Step 1 (a negative control.)

Another way in which to test whether chimers and internally deleted tail fiber proteins retain the ability to multimerize and associate with their appropriate partners is done in vivo. The assay detects the ability of such chimers and deleted proteins to compete with normal phage parts for assembly, thus reducing the burst size of a wild-type phage infecting the same host cell in which the chimers or deleted proteins are recombinantly expressed. Thus, expression from an expression vector encoding the chimer or deleted protein is induced inside a cell, which cell is then infected by a wild-type phage. Inhibition of wild-type phage production demonstrates the ability of the recombinant chimer or protein to associate with the appropriate tail fiber proteins of the phage.

The above-described methods are used, alone and in combination, in the design and production of different types of modified tail fiber proteins. For example, a preliminary screen of a large number of bacterial colonies for those expressing a properly multimerized protein will identify positive colonies, which can then be individually tested by in vitro complementation.

Non-limiting examples of novel proteins that are encompassed by the present invention include:

1) Internally deleted gp34, 36, and 37 polypeptides (See Example 1 below);

2) A C-terminally truncated gp36 fused to the N-terminus of N-terminally truncated gp37, i.e, a fusion between gp36 and gp37 in which gp36 is N-terminal to gp37;

3) A fusion between gp36 and gp37 in which gp37 is N-terminal to gp36 (i.e., in reverse of the natural order), termed herein “gp37-36 chimer” (See Example 2 below);

4) A fusion between gp34 and gp36 in which gp36 is N-terminal to gp34 (i.e., in reverse of the natural order), termed herein “gp36-34 chimer”;

5) A variant of gp36 in which the C-terminus is mutated such that it lacks the capability to interact with (and multimerize in response to) the N-terminus of wild-type P37, termed herein “gp36*”;

6) A variant of gp37 in which the N-terminus is mutated such that it forms a P37 that lacks the capability to interact with the C-terminus of wild-type gp36, termed herein “*P37”;

7) Variants of gp36* and *P37 that can interact with each other, but not with gp36 or P37.

8) A variant “P37-36 chimer” in which the gp36 moiety is derived from the variant as in 5), i.e., “P37-36*”. (For 5-8, See Example 3 below.)

9) A variant “P37-36 chimer” in which the gp37 moiety is derived from the variant as in 6) above, i.e., “*P37-36”.

10) A variant P37-36 chimer, *P37-P36*, in which the gp36 and gp37 moieties are derived from the variants in 7).

11) A fusion between gp36 and gp34 in which gp36 sequences are placed N-terminal to gp34, the multimer of which is termed herein “P36-34 chimer”;

12) Variants of gp35 that form average angles different from 137° or 158° (the native angle) e.g., less than about 125° or more than about 145° under conditions wherein the wild-type gp35 protein forms an angle of 137° when combined with the P34 and P36-P37 multimers, and/or exhibit more or less flexibility-than the native polypeptide;

13) Variants of gp34, 35, 36 and 37 that exhibit thermolabile interactions or other variant specific interactions with their cognate partners; and

14) Variants of gp37 in which the C-terminal domain of the polypeptide is modified to include sequences that confer specific binding properties on the entire molecule, e.g., sequences derived from avidin that recognize biotin, sequences derived from immunoglobulin heavy chain that recognize Staphylococcal A protein, sequences derived from the Fab portion of the heavy chain of monoclonal antibodies to which their respective Fab light chain counterparts could attach and form an antigen-binding site, immunoactive sequences that recognize specific antibodies, or sequences that bind specific metal ions. These ligands may be immobilized to facilitate purification and/or assembly.

In specific embodiments, the chimers of the invention comprise a portion consisting of at least the first 10 (N-terminal) amino acids of a first tail fiber protein fused via a peptide bond to a portion consisting of at least the last 10 (C-terminal) amino acids of a second tail fiber protein. The first and second tail fiber proteins can be the same or different proteins. In another embodiment, the chimers comprise an amino acid portion in the range of the first 10-60 amino acids from a tail fiber protein fused to an amino acid portion in the range of the last 10-60 amino acids from a second tail fiber protein. In another embodiment, each amino acid portion is at least 20 amino acids of the tail fiber protein. The chimers comprise portions, i.e., not full-length tail fiber proteins, fused to one another. In a preferred aspect, the first tail fiber protein portion of the chimer is from gp37, and the second tail fiber protein portion is from gp36. Such a chimer (gp37-36 chimer), after oligomerization to form P37-36, can polymerize to other identical oligomers. A gp36-34 chimer, after oligomerization to form P36-34, can bind to gp35, and this unit can then polymerize. In another embodiment, the first portion is from gp37, and the second portion is from gp34. In a preferred aspect, the chimers of the invention are made by insertions or deletions within a β turn of the β structure of the tail fiber proteins. Most preferably, insertions into a tail fiber sequence, or fusing to another tail fiber protein sequence, (preferably via manipulation at the recombinant DNA level to produce the desired encoded protein) is done so that sequences in β turns on the same edge of the β-sheet are joined.

In addition to the above-described chimers, nanostructures of the invention can also comprise tail fiber protein deletion constructs that are truncated at one end, e.g., are lacking an amino- or carboxy- end (of at least 5 or 10 amino acids) of the molecule. Such molecules truncated at the amino-terminus, e.g., of truncated gp37, gp34, or gp36, can be used to “cap” a nanostructure, since, once incorporated, they will terminate polymerization. Such molecules preferably comprise a fragment of a tail fiber protein lacking at least the first 10, 20, or 60 amino terminal amino acids.

In order to change the length of the rod component proteins as desired, portions of the same or different tail fiber proteins can be inserted into a tail fiber chimer to lengthen the rod, or be deleted from a chimer, to shorten the rod.

Assembly of Individual Rod Structural Units Into Nanostructures

Expression of proteins structural units in E. coli as described above results in the synthesis of large quantities of protein, and allows the simultaneous expression and assembly of different components in the same cells. The methods for scale-up of recombinant protein production are straightforward and widely known in the art, and many standard protocols can be used to recover native and modified tail fiber proteins from a bacterial culture.

In a preferred embodiment, native (nonrecombinant) gp35 is isolated for use by growing up a bacteriophage T4 having an amber mutation in-gene 36, in a su⁰ bacterial strain (not an amber suppressor), and isolating gp35 from the resulting culture by standard methods.

P34, P36-P37, P37, and chimers derived from them are purified from E. coli cultures as mature multimers. Gp35 and variants thereof are purified as monomers. Purification is achieved by the following procedures or combinations thereof, using standard methods: 1) chromatography on molecular sieve, ion-exchange, and/or hydrophobic matrices; 2) preparative ultracentrifugation; and 3) affinity chromatography, using as the immobilized ligand specific antibodies or other specific binding moieties. For example, the C-terminal domain of P37 binds to the lipopolysaccharide of E. coli B. Other T4-like phages have P37 analogues that bind other cell surface components such as OmpF or TSX protein. Alternatively, if the proteins have been engineered to include heterologous domains that act as ligands or binding sites, the cognate partner is immobilized on a solid matrix and used in affinity purification. For example, such a heterologous domain can be biotin, which binds to a streptavidin-coated solid phase.

Alternatively, several components are co-expressed in the same bacterial cells, and sub-assemblies of larger nanostructures are purified subsequent to limited in vivo assembly, using the methods enumerated above.

The purified components are then combined in vitro under conditions where assembly of the desired nanostructure occurs at temperatures between about 4° C. and about 37° C., and at pHs between about 5 and about 9. For a given nanostructure, optimal conditions for assembly (i.e., type and concentration of salts and metal ions) are easily determined by routine experimentation, such as by changing each variable individually and monitoring formation of the appropriate products.

Alternatively, one or more crude bacterial extracts may be prepared, mixed, and assembly reactions allowed to proceed prior to purification.

In some cases, one or more purified components assemble spontaneously into the desired structure, without the necessity for initiators. In other cases, an initiator is required to nucleate the polymerization of rods or sheets. This offers the advantage of localizing the assembly process (i.e., if the initiator is immobilized or otherwise localized) and of regulating the dimensions of the final structure. For example, rod components that contain a functional P36 C-terminus require a functional P37 N-terminus to initiate rod formation stoichiometrically; thus, altering the relative amount of initiator and rod component will influence the average length of rod polymer. If the ratio is n, the average rod will be approximately (P37-36)n-N-terminus P37-P37 C-terminus.

In the method of the invention, the final nanostructure is composed of two or more protein structural units that cannot self-assemble individually but only in combination with each other. In this situation, alternating cycles of assembly can be staged to produce final products of precisely defined structure. (see Example 6B below.)

When an immobilized initiator is used, it may be desirable to remove the polymerized unit from the matrix after staged assembly. For this purpose specialized initiators may be engineered so that the interaction with the first rod component is rendered reversibly thermolabile (see Example 5 below). In this way, the polymer can be easily separated from the matrix-bound initiator, thereby permitting: 1) easy preparation of stock solutions of uniform parts or subassemblies, and 2) re-use of the matrix-bound initiator for multiple cycles of polymer initiation, growth, and release.

In an embodiment in which a nanostructure is assembled that is attached to a solid matrix via gp34 (or P34), one way in which to detach the nanostructure to bring it into solution is to use a mutant (thermolabile) gp34 that can be made to detach upon exposure to a higher temperature (e.g., 40° C.). Such a mutant gp34, termed T4 tsB45, having a mutation at its C-terminal end such that P34 attaches to the distal tail fiber half at 30° C. but can be separated from it in vitro by incubation at 40° C. in the presence of 1% SDS (unlike wild-type T4 which are stable under these conditions), has been reported (Seed, 1980, Studies of the Bacteriophage T4 Proximal Half Tail Fiber, Ph. D. Thesis, California Institute of Technology), and can be used.

Proteins which catalyze the formation of correct (lowest energy) stable secondary (2°) structure of proteins are called chaperone proteins. (Often, especially in globular proteins, this stabilization is aided by tertiary structure, e.g., stabilization of β-sheets by their interaction in β-barrels or by interaction with .alpha.-helices). Normally chaperonins prevent intrachain or interchain interactions which would produce untoward metastable folding intermediates and prevent or delay proper folding. There are two known accessory proteins, gp57 and gp38, in the morphogenesis of T4 phage tail fibers which are sometimes called chaperonins because they are essential for proper maturation of the protein oligomers but are not present in the final structures.

Gp57, probably in conjunction with some membrane protein(s), has the role of juxtaposing (and aligning) and/or initiating the folding of 2 or 3 identical gp37 molecules. The aligned peptides then zip up (while mutually stabilizing their nascent β-structures) to form a beam, without further interaction with gp57. Gp57 acts in T4 assembly not only for oligomerization of gp37 but also for gp34 and gp12.

Structural Components for Self Assembly of Beams In Vitro

Alternatively to starting the polymerization of chimers with the use of a preformed chimeric or natural oligomeric unit called an initiator produced in vivo, molecules (preferably peptides) that can self-assemble can be produced as fusion proteins, fused to the N- or C-terminus of tail fiber variants of the invention (chimers, deletion/insertion constructs) to align their ends and thus to facilitate their subsequent unaided folding into oligomeric, stable β-folded rod-like (beam) units in vitro, in the absence of the normally required chaperonin proteins (e.g., gp57) and host cell membrane proteins.

As an illustration, consider the P37 unit as an initiator of gp37-36 oligomerization and polymerization. Normally, proper folding of gp37 to a P37 initiator requires a phage infected cell membrane, and two chaperone proteins, gp38 and gp57. In a preferred embodiment, the need for gp38 can be obviated by use of a mutation, ts3813 (a duplication of 7 residues just downstream of the transition zone of gp37) which suppresses gene 38 (Wood, W. B., F. A. Eiserling and R. A. Crowther, 1994, “Long Tail Fibers: Genes, Proteins, Structure, and Assembly,” in Molecular Biology of Bacteriophage T4, (Jim D. Karam, Editor) American Society for Microbiology, Washington, D.C., pp 282-290). If a moiety that self-assembles into a multimer or trimer or other oligomer (“self-assembling moiety”) is fused to a C-terminal deletion of gp37 downstream or upstream of the transition region [the transition region is a conserved 17 amino acid residue region in T4-like tail fiber proteins where the structure of the protein narrows to a thin fiber; see Henning et al., 1994, “Receptor recognition by T-even-type coliphages,” in Molecular Biology of Bacteriophage T4, Karam (ed.), American Society for Microbiology, Washington, D.C., pp. 291-298; Wood et al., 1994, “Long tail fibers: Genes, proteins, structure, and assembly,” in Molecular Biology of Bacteriophage T4, Karam (ed.), American Society for Microbiology, Washington, D.C., pp. 282-290], when it is expressed, the self-assembling moiety will oligomerize in parallel and thus align the fused gp37 peptides, permitting them to fold in vitro, in the absence of other chaperonin proteins.

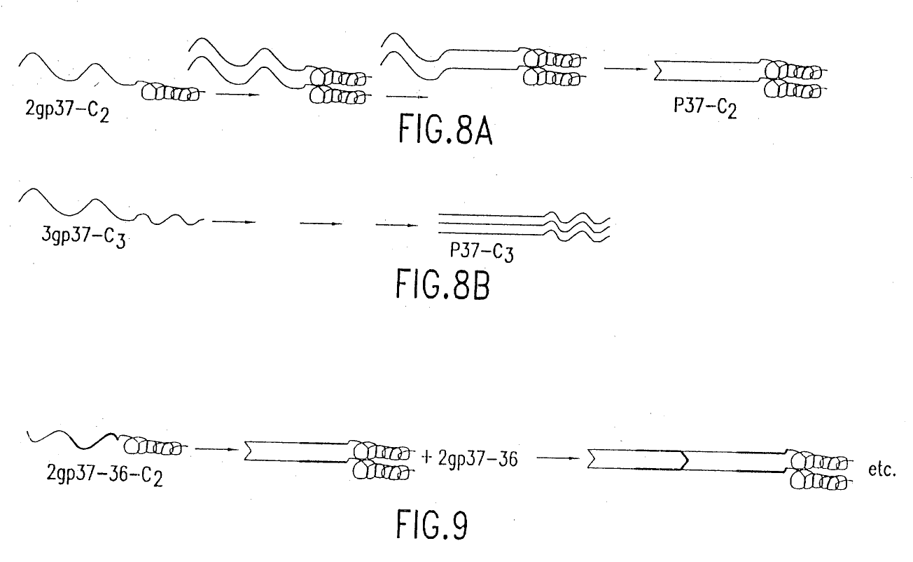

If P37 is a dimer (FIG. 8A), the self-assembling moiety can be a self dimerizing peptide such as the leucine zipper, made from residues 250-281 from the yeast transcription factor, GCN4 (E. K. O'Shea, R. Rutkowski and P. S. Kim, Science 243:538, 1989) or the self dimerizing mutant leucine zipper peptide, pWL in which the a positions are substituted with isoleucine and the d positions with leucine (Harbury P. B., T. Zhang, P. S. Kim and T. Alper. 1993. A Switch Between Two-, Three-, and Four-Stranded Coiled Coils in GCN4 Leucine Zipper Mutants. Science, 262:1401-1407). If P37 is a trimer (FIG. 8B), the self-assembling moiety can be a self trimerizing mutant leucine zipper peptide, p11 in which both the a and d positions are substituted with isoleucine (Harbury P. B., et al. ibid). Alternatively, a collagen peptide can be used as the self-assembling moiety, such as that described by Bella et al. (J. Bella, M. Eaton, B. Brodsky and H. M. Berman. 1994. Crystal and Molecular Structure of a Collagen-Like Peptide at 1.9 .ANG. Resolution. Science, 226:75-81), which self aligns by an inserted specific non repeating alanine residue near the center.

Self-assembling moieties can be used to make initiators for polymerizations in the absence of the normal initiators. For example, to create an initiator for oligomerization and polymerization of the chimeric monomer, gp37-36, gp37-36-C₂ can be used as illustrated in FIG. 9. (C₂ means that a dimer forming peptide is fused to the C-terminus of the gp36 moiety. This is used if the beam is a dimeric structure. Otherwise C₃—a trimer forming peptide fused to the C-terminus—would be used.) Furthermore, use of the E. coli lac repressor N-terminus, e.g., which associates as a tetramer, with two coils facing in each direction could join two multimers (or polymers of multimers) end to end, either at their N- or C-termini depending upon which end the self-assembling peptides were placed. They could also join N- to C-termini. In any case, alone, they could only form a dimer, each end of which would be extensible by adding an appropriate chimer monomer (as shown for the simpler case in FIG. 9).

In an alternative embodiment, the self-assembling moiety can be fused to the N-termini of the chimer. In a specific embodiment, the self-assembling moiety is fused to at least a 10 amino acid portion of a T-even-like tail fiber protein.

A self assembling moiety that assembles into a heter-oligomer can also be used. For example, if polymerization between beams is directed by the surface of a dimeric cross-β surface, addition of a heterodimeric unit with one surface which does not promote further polymerization would be very useful to cap the penultimate unit and thus terminate polymerization. If the two types of coiled regions of the self-assembling moiety are much more attractive to each other that to themselves, then all of the dimers will be heterodimers. Such is the case for the N-terminal Jun and Fos leucine zipper regions.

A further advantage to such heterodimeric units is the ability to stage polymerization and thus build one unit (or one surface in a 2D array) at a time. For example, suppose surface A attaches to B but neither attaches to itself ([A⇄B] is used to symbolize this type of interaction). Mix A/A and B/B₀ (B₀ is attached to a matrix for easy purification). This will form B₀/B-A/A. Now wash out A/A and add B/B. The construct is now B₀/B-A/A-B/B. Now add A/A₀. The construct is now B₀/B-A/A-B/B-A/A₀ and no more beams can be added. There are of course many other possibilities.

Applications

The uses of the nanostructures of the present invention are manifold and include applications that require highly regular, well-defined arrays of fibers, cages, or solids, which may include specific attachment sites that allow them to associate with other materials.

In one embodiment, a three-dimensional hexagonal array of tubes is used as a molecular sieve or filter, providing regular vertical pores of precise diameter for selective separation of particles by size. Such filters can be used for sterilization of solutions (i.e., to remove microorganisms or viruses), or as a series of molecular-weight cut-off filters. In this case, the protein components of the pores may be modified so as to provide specific surface properties (i.e., hydrophilicity or hydrophobicity, ability to bind specific ligands, etc.). Among the advantages of this type of filtration device is the uniformity and linearity of pores and the high pore to matrix ratio.

In another embodiment, long one-dimensional fibers are incorporated, for example, into paper or cement or plastic during manufacture to provide added wet and dry tensile strength.

In still another embodiment, different nanostructure arrays are impregnated into paper and fabric as anti-counterfeiting markers. In this case, a simple color-linked antibody reaction (such as those commercially available in kits) is used to verify the origin of the material. Alternatively, such nanostructure arrays could bind dyes or other substances, either before or after incorporation to color the paper or fabrics or modify their appearance or properties in other ways.

Kits

The invention also provides kits for making nanostructures, comprising in one or more containers the chimers and deletion constructs of the invention. For example, one such kit comprises in one or more containers purified gp35 and purified gp36-34 chimer. Another such kit comprises purified gp37-36 chimer.

The following examples are intended to illustrate the present invention without limiting its scope.

In the examples below, all restriction enzymes, nucleases, ligases, etc. are commercially available from numerous commercial sources, such as New England Biolabs (NEB), Beverly, Mass.; Life Technologies (GIBCO-BRL), Gaithersburg, Md.; and Boehringer Mannheim Corp. (BMC), Indianapolis, Ind.

### EXAMPLE 1

Design, Construction and Expression of Internally Deleted P37

The gene encoding gp37 contains two sites for the restriction enzyme Bgl II, the first cleavage occurring after nucleotide 293 and the second after nucleotide 1486 (the nucleotides are numbered from the initiator methionine codon ATG.) Thus, digestion of a DNA fragment encoding gp37 with BglII, excision of the intervening fragment (nucleotides 294-1485) and re-ligation of the 5′ and 3′ fragments results in the formation of an internally deleted gp37, designated ΔP37, in which arginine-98 is joined with serine-497.

The restriction digestion reaction mix contains:

| | |
|---|---|
| gp37 plasmid DNA (1 μg/μl) | 2 μl |
| NEB buffer #2 (10X) | 1 μl |
| H₂O | 6 μl |
| Bgl II (10 U/μl) | 1 μl |

The gp37 plasmid signifies a pT7-5 plasmid into which gene 37 has been inserted in the multiple cloning site, downstream of a good ribosome binding site and of gene 57 to chaperon the dimerization. The reaction is incubated for 1 h at 37° C. Then, 89 μl of T4 DNA ligase buffer and 1 μl of T4 DNA ligase are added, and the reaction is continued at 16° C. for 4 hours. 2 μl of Stu I restriction enzyme are then added, and incubation continued at 37° C. for 1 h. (The Stu I restriction enzyme digests residual plasmids that were not cut by Bgl II in the first step, reducing their transformability by about 100-fold.)

The reaction mixture is then transformed into E. coli strain BL21, obtained from Novagen, using standard procedures. The transformation mixture is plated onto nutrient agar containing 100 μg/ml ampicillin, and the plates are incubated overnight at 37° C.

Colonies that appear after overnight incubation are picked, and plasmid DNA is extracted and digested with Bgl II as above. The restriction digests are resolved on 1% agarose gels. A successful deletion is evidenced by the appearance after gel electrophoresis of a new DNA fragment of 4.2 kbp, representing the undeleted part of gene 37 which is still attached to the plasmid and which re-formed a BglII site by ligation. The 1.2 kbp DNA fragment bounded by BglII sites in the original gene is no longer in the plasmid and so is missing from the gel.

Plasmids selected for the predicted deletion as above are transformed into E. coli strain BL21(DE3). Transformants are grown at 30° C. until the density (A₆₀₀) of the culture reaches 0.6. IPTG is then added to a final concentration of 0.4 mm and incubation is continued at 30° C. for 2 h, after which the cultures are chilled on ice. 20 μl of the culture is then removed and added to 20 μl of a two-fold concentrated “cracking buffer” containing 1% sodium dodecyl sulfate, glycerol, and tracking dye. 15 μl of this solution are loaded onto a 10% polyacrylamide gel; a second aliquot of 15 μl is first incubated in a boiling water bath for 3 min and then loaded on the same gel. After electrophoresis, the gel is fixed and stained. Expression of the deleted gp37 is evidenced by the appearance of a protein species migrating at an apparent molecular mass of 65-70,000 daltons in the boiled sample. The extent of dimerization is suggested by the intensity of higher-molecular mass species in the unboiled sample and/or by the disappearance of the 65-70,000 dalton protein band.

The ability of the deleted polypeptide to multimerize appropriately is directly evaluated by testing its ability to be recognized by an anti-P37 antiserum that reacts only with mature P37 dimers, using a standard protein immunoblotting procedure.

An alternative assay for functional multimerization of the deleted P37 polypeptide (also referred to as ΔP37) is its ability to complement in vivo a T4 37⁻ phage, by first inducing expression of the ΔP37 and then infecting with the T4 mutant, and detecting progeny phage.

A ΔP37 was prepared as described above, and found capable of complementing a T4 37⁻ phage in vivo.

### EXAMPLE 2

Design, Construction and Expression of a gp37-36 Chimer

The starting plasmid for this construction is one in which the gene encoding gp37 is cloned immediately upstream (i.e., 5′) of the gene encoding gp36. The plasmid is digested with Hae III, which deletes the entire 3′ region of gp37 DNA downstream of nucleotide 724 to the 3′ terminus, and also removes the 5′ end of gp36 DNA from the 5′ terminus to nucleotide 349. The reaction mixture is identical to that described in Example 1, except that a different plasmid DNA is used, and the enzyme is HaeIII. Ligation using T4 DNA ligase, bacterial transformation, and restriction analysis are also performed as in Example 1. In this case, excision of the central portion of the gene 37-36 insert and religation reveals a novel insert of 346 in-frame codons, which is cut only once by HaeIII (after nucleotide 725). The resulting construct is then expressed in E. coli BL21(DE3) as described in Example 1.

Successful expression of the gp37-36 chimer is evidenced by the appearance of a protein product of about 35,000 daltons. This protein will have the first 242 N-terminal amino acids of gp37 fused to the final 104 C-terminal amino acids of gp36 (numbered 118-221.) The utility of this chimer depends upon its ability to dimerize and attach end-to-end. That is, carboxy termini of said polypeptide will have the capability of interacting with the amino terminus of the P37 protein dimer of bacteriophage T4 and to form an attached dimer, and the amino terminus of the dimer of said polypeptide will have the capability of interacting with other said chimer polypeptides. This property can be tested by assaying whether introduction of ΔP37 initiates multimerization and polymerization. Alternatively, polyclonal antibodies specific to P36 dimer may be used to detect P36 subsequent to initiation of dimerization by ΔP37.

A gp37-36 chimer was prepared similarly to the procedures described above, except that the restriction enzyme TaqI was used instead of HaeIII. Briefly, the 5′ fragment resulting from TaqI digestion of gene 37 was ligated to the 3′ fragment resulting from TaqI digestion of gene 36. This produced a construct encoding a gp37-36 chimer in which amino acids 1-48 of gp37 were fused to amino acids 100-221 of gp36. This construct was expressed in E. coli BL21(DE3), and the chimer was detected as an 18 kD protein. This gp37-36 chimer was found to inhibit the growth of wild type T4 when expression of the gp37-36 chimer was induced prior to infection (in an in vitro phage inhibition assay).

### EXAMPLE 3

Mutation of the GP37-36 Chimer to Produce Complementary Suppressors

The goal of this construction is to produce two variants of a multimerizable P37-36 chimer: One in which the N-terminus of the polypeptide is mutated (A, designated *P37-36) and one in which the C-terminus of the polypeptide is mutated (B, designated P37-36*). The requirement is that the mutated *P37 N-terminus cannot form a joint with the wild-type P36 C-terminus, but only with the mutated *P36 N-terminus. The rationale is that A and B each cannot polymerize independently (as the parent P37-36 protein can), but can only associate with each other sequentially (i.e., P37-36*+*P37-36→P37-36*−*P37-36).

A second construct, *p37-P36*, is formed by recombining *P37-36 and P37-36* in vitro. When the monomers *gp37-36* and gp37-36 are mixed in the presence of P37 initiator, gp37-36 would multimerize and polymerize to (P37-36)n; similarly, *P37 would only catalyze the polymerization of *gp37-36* to (*P37-36*)n. In this case, the two chimers could be of different size and different primary sequence with different potential side-group interactions, and could initiate attachment at different surfaces depending on the attachment specificity of P37.

The starting bacterial strain is a su⁰ strain of E. coli (which lacks the ability to suppress amber mutations). When this strain is infected with a mutant T4 bacteriophage containing amber mutations in genes 35, 36, and 37, phage replication is incomplete, since the tail fiber proteins cannot be synthesized. When this strain is first transformed with a plasmid that directs the expression of the wild type gp35, gp36and gp37 genes and induced with IPTG, and subsequently plated onto agar media containing mutant phage, the mutant phage can infect the growing bacterial colony and infectious phage particles are produced; this is evidenced by the appearance of “nibbled” colonies. Nibbled colonies do not appear round, with smooth edges, but rather have sectors missing. This is caused by attack of a microcolony by a single phage, which replicates and prevents the growth of the bacteria in the missing sector.

For the purposes of this construction, the 3′-terminal region of gene 36 (corresponding to the C-terminal region of gp36) is mutagenized with randomly doped oligonucleotides. Randomly doped oligonucleotides are prepared during chemical synthesis of oligonucleotides, by adding a trace amount (up to a few percent) of the other three nucleotides at a given position, so that the resulting oligonucleotide mix has a small percentage of incorrect nucleotides at that position. Incorporation of such oligonucleotides into the plasmid will result in random mutations (Hutchison et al., Methods.Enzymol. 202:356, 1991).

The mutagenized population of plasmids (containing, however, unmodified genes 36 and 37), is then transformed into the su⁰ bacteria, followed by infection with the mutant T4 phage as above. In this case, the appearance of non-“nibbled” colonies indicates that the mutated gp36 C-termini can no longer interact with wild type P37 to form functional tail fibers. The putative gp36* phenotypes found in such non-nibbled colonies are checked for lack of multimeric N-termini by appropriate immunospecificity as outlined above, and positive colonies are used as source of plasmid for the next step.

Several of these mutated plasmids are recovered and subjected to a second round of mutagenesis, this time using doped oligonucleotides that introduce random mutations into the N-terminal region of gp37 present on the same plasmid. Again, the (now doubly) mutagenized plasmids are transformed into the ⁰ strain of E. coli and transformants are infected with the mutant T4 phage. At this stage, bacterial plates are screened for the re-appearance of “nibbled” colonies. A nibbled colony at this stage indicates that the phage has replicated by virtue of suppression of the non-functional gp36* mutation(s) by the *P37 mutation. In other words, such colonies must contain novel *P37 polypeptides that have now acquired the ability to interact with the P36* proteins encoded on the same plasmid.

The *P37-36 and P37-36* paired suppressor chimers (A and B as above) are then constructed in the same manner as described in Example 2. In this case, however, *P37 is used in place of wild type P37 and P36* is used in place of wild type P36. A *P37-36* chimer can now be made by restriction of *P37-36 and P37-36* and religation in the recombined order. The *P37-36* can be mixed with the P37-36 chimer, and the polymerization of each can be accomplished independently in the presence of the other. This is useful when the rod-like central portion of these chimers have been modified in different ways.

### EXAMPLE 4

Design, Construction and Expression of a gp36-34 Chimer

The starting plasmid for this construction is one in which the vector containing gene 57 and the gene encoding gp36 is cloned immediately upstream (i.e., 5′) of the gene encoding gp34. The plasmid is digested with NdeI, which cuts after bp 219 of gene 36 and after bp 2594 of gene 34, thereby deleting the final 148 C-terminal codons from the pg36 moiety and the first 865 N-terminal codons from the gp34 moiety. The reaction mixture is identical to that described in Example 1, except that a different plasmid DNA is used, and the enzyme used is NdeI (NEB). Ligation using T4 DNA ligase, bacterial transformation, and restriction analysis are also performed as in Example 1. This results in a new hybrid gene encoding a protein of 497 amino acids (73 N-terminal amino acids of gp36 and 424 C-terminal amino acids of gp34, numbered 866-1289.)

As an alternative, the starting plasmid is cut with SphI at bp 648 in gene 34, and the Exo-Size Deletion Kit (NEB) is used to create deletions as described above.

The resulting construct is then expressed in E. coli BL21(DE3) as described in Example 1. Successful expression of the gp36-34 chimer is evidenced by the appearance of a protein product of about 55,000 daltons. Preferably, the amino termini of the polypeptide homodimer have the capability of interacting with the gp35 protein, and then the carboxy termini have the capability of interacting with other attached gp35 molecules. Successful formation of the dimer can be detected by reaction with anti-P36 antibodies or by attachment of gp35 or by the in vitro phage inhibition assay described in Example 2.

### EXAMPLE 5

Isolation of Thermolabile Proteins for Self-Assembly

Thermolabile structures can be utilized in nanostructures for: a) initiation of chimer polymerization (e.g., gp37-36) at low temperature and subsequent inactivation of and separation from the initiator at high temperature; b) initiation of angle formation between P36 and gp35 (e.g., variants of gp35 that have thermolabile attachment sites for P36 N-termini or P34 C-termini, a variant P36 that forms a thermolabile attachment to gp35, and a variant P34 with a thermolabile C-terminal attachment site.) Thermolability may be reversible, permitting reattachment of the appropriate termini when the lower temperature is restored, or it may be irreversible.

To create a variant gp37 that permits heat induced separation of the P36-P37 junction, the 5′ end of gp37 DNA is randomly mutagenized using doped oligonucleotides as described above. The mutagenized DNA fragment is then recombined into T4 phage by infection of the cell containing the mutagenized DNA by a T4 phage containing two amber mutations flanking the mutagenized region. Following a low-multiplicity infection, non-amber phage are selected at low temperature on E. coil su⁰ at 30° C. The progeny of these plaques are resuspended in buffer and challenged by heating at 60° C. At this temperature, wild-type tail fibers remain intact and functional, whereas the thermolabile versions release the terminal P37 units and thus render those phage non-infectious.

At this stage, wild type phage are removed by: 1) adsorbing the wild type phage to sensitive bacteria and sedimenting (or filtering out) the bacteria with the adsorbed wild type phage; or 2) reacting the lysate with anti-P37 antibody, followed by immobilized Protein A and removal of adsorbed wild type phage. Either method leaves the noninfectious mutant phage particles in the supernatant fluid or filtrate, from which they can be recovered. The non-infectious phage lacking terminal P37 moieties (and probably the rest of the tail fibers as well) are then urea treated with 6M urea, and mixed with bacterial spheroplasts to permit infection at low multiplicity whereupon they replicate at low temperature and release progeny. Alternatively, infectious phage are reconstituted by in vitro incubation of the mutant phage with wild type P37 at 30° C.; this is followed by infection of intact bacterial cells using the standard protocol. The latter method of infection specifically selects mutant phage in which the thermolability of the P36-P37 junction is reversible.

Using either method, the phage populations are subjected to multiple rounds of selection as above, after which individual phage particles are isolated by plaque purification at 30° C. Finally, the putative mutants are evaluated individually for the following characteristics: 1) loss of infectivity after incubation at high temperatures (40-60° C.), as measured by a decrease in titer; 2) loss of P37 after incubation at high temperature, as measured by decrease in binding of P37-specific antibody to phage particles; and 3) morphological changes in the tail fibers after incubation at high temperatures, as assessed by electron microscopy.

After mutants are isolated and their phenotypes confirmed, the P37 gene is sequenced. If the mutations localize to particular regions or residues, those sequences are targeted for site-directed mutagenesis to optimize the desired characteristics.

Finally, the mutant gene 37 is cloned into expression plasmids and expressed individually in E. coli as in Example 1. The mutant-P37 dimers are then purified from bacterial extracts and used in vitro assembly reactions.

In a similar fashion, mutant gp35 polypeptides can be isolated that exhibit a thermolabile interaction with the N-terminus of P36 or the C-terminus of P34. For thermolabile interaction with P34, phage are incubated at high temperature, resulting in the loss of the entire distal half of the tail fiber (i.e., gp35-P36-P37). The only difference in the experimental protocol is that, in this case, 1) random mutagenesis is performed over the entire gp35 gene; 2) wild-type phage (and distal half-fibers from thermolabile mutants) are separated from thermolabile mutant phage that have been inactivated at high temperature (but still have proximal half tail fibers attached) by precipitating both the distal half-fibers and the phage particles containing intact tail fibers with any of the anti-distal half tail-fiber antibodies followed by Staphylococcal A-protein beads; 3) the mutant phage remaining in the supernatant are reactivated by incubation at low temperature with bacterial extracts containing wild type intact distal half fibers; and 4) stocks of thermolabile gene, 35 mutants grown at 30° C. can be tested for reversible thermolability by inactivation at 60° C. and reincubation at 30° C. Inactivation is performed on a concentrated suspension of phage, and reincubation at 30° C. is performed either before or after dilution. If phage are successfully reactivated before, but not after, dilution, this indicates that their gp35 is reversibly thermolabile.

To create a gene 36 mutation with a thermolabile gp35-P36 linkage, the C-terminus of gene 36 is mutagenized as described above, and the mutant selected for reversibility. An alternative is to mutagenize gp35 to create a gene 35 mutant in which the gp35-P36 linkage will dissociate at 60° C. In this case, incubation with anti-gp35 antibodies can be used to precipitate the phage without P36-P37 and thus to separate them from the wild-type phage and distal half-tail fibers (P36-P37), since the variant gp35 will remain attached to P34.

### EXAMPLE 6

Assembly of One-Dimensional Rods

A. Simple Assembly: The P37-36 chimer described in Example 2 is capable of self-assembly, but requires a P37 initiator to bind the first unit of the rod. Therefore, a P37 or a ΔP37 dimer is either attached to a solid matrix or is free in solution to serve as an initiator. If the initiator is, attached to a solid matrix, a thermolabile P37 dimer is preferably used. Addition of an extract containing gp37-36, or the purified gp37-36 chimer, results in the assembly of linear multimers of increasing length. In the matrix-bound case, the final rods are released by a brief incubation at high temperature (40-60° C., depending on the characteristics of the particular thermolabile P37 variant.)

The ratio of initiator to gp37-36 can be varied, and the size distribution of the rods is measured by any of the following methods: 1) Size exclusion chromatography; 2) Increase in the viscosity of the solution; and 3) Direct measurement by electron microscopy.

B. Staged assembly: The P37-36 variants *P37-36 and P37-36* described in Example 3 cannot self-polymerize. This allows the staged assembly of rods of defined length, according to the following protocol:

1. Attach initiator P37 (preferably thermolabile) to a matrix.

2. Add excess *gp37-36 to attach and oligomerize as P37-36 homooligomers to the N-terminus of P37.

3. Wash out unreacted *gp37-36 and flood with gp37-36*.

4. Wash out unreacted gp37-36* and flood with excess *gp37-36.

5. Repeat steps 2-4, n-1 times.

6. Release assembly from matrix by brief incubation at high temperature as above.

The linear dimensions of the protein rods in the batch will depend upon the lengths of the unit heterochimers and the number of cycles (n) of addition. This method has the advantage of insuring absolute reproducibility of rod length and a homogenous, monodisperse size distribution from one preparation to another.

### EXAMPLE 7

Staged Assembly of Polygons

The following assembly strategy utilizes gp35 as an angle joint to allow the formation of polygons. For the purpose of this example, the angle formed by gp35 is assumed to be 137°. The rod unit comprises the P36-34 chimer described in Example 4, which is incapable of self-polymerization. The P36-34 homodimer is made from a bacterial clone in which both gp36-34 and gp57 are expressed. The gp57 can chaperone the homodimerization of gp36-34 to P36-34.

1. Initiator: The incomplete distal half fiber P36-37 is attached to a solid matrix by the P37 C-terminus. Thermolabile gp35 as described in Example 5 is then added to form the intact initiator.

2. Excess P36-34 chimer is added to attach a single P36-34. Following binding to the matrix via gp35, the unbound chimer is washed out.

3. Wild-type (i.e., non-thermolabile) gp35 is then added in excess. After incubation, the unbound material is washed out.

4. Steps 2 and 3 are repeated 7-8 times.

5. The assembly is released from the matrix by brief incubation at high temperature.

The released polymeric rod, 8 units long, will form a regular 8-sided polygon, whose sides comprise the P36-34 dimer and whose joints comprise the wild-type gp35 monomer. However, there will be some multimers of these 8 units bound as helices. When a unit does not close, but instead adds another to its terminus, the unit cannot close further and the helix can build in either direction. The direction of the first overlap also determines the handedness of the helix. Ten (or seven)-unit rods may form helices more frequently than polygons since their natural angles are 144° (or 128.6°). The likelihood of closure of a regular polygon depends not only on the average angle of gp35 but also on its flexibility, which can be further manipulated by genetic or environmental modification.

The type of polygon that is formed using this protocol depends upon the length of rod units and the angle formed by the angle joint. For example, alternating rod units of different sizes can be used in step 2. In addition, variant gp35 polypeptides that form angles different than the natural angle of 137° can be used, allowing the formation of different regular polygons. Furthermore, for a given polygon with an even number of sides and equal angles, the sides in either half can be of any size provided the two halves are symmetric.

### EXAMPLE 8

PCR analysis was used to screen spontaneous pseudorevertants of a gene 37 amber mutation (amA481), and a phage was selected that appeared to have approximately 1 kb of DNA deleted from the middle of gene 37. This gene codes for the protein forming the distal end of the tail fibers, and its C terminus forms the phage receptor. Sequence analysis confirmed that a single contiguous segment of DNA coding for 346 of 1,026 amino acid residues (34%) was deleted in this phage, which was designated SΔ1 (spontaneous deletion 1). Table 1 shows the protein sequences of the deletion junctions and the corresponding wild-type protein. The deleted region begins at amino acid 73, which is 23 residues downstream from the conserved N-terminal domain of P37. This conserved region is thought to form the stiff butt end joint with the P36 C-terminal conserved domain. (Riede, I. , Drexler, K. & Eschbach, M. -L. (1985) Nucleic Acids Res. 13, 605-616.) Thus, this deletion falls completely within the P37 rod-like region. Phage carrying the SΔ1 mutation produce plaques of normal size and appearance indicating that they are able to infect and grow normally. We also measured the adsorption rate of the SΔ1 phage (a measure of the rate of irreversible binding to the cell surface) and found that it was the same as wild-type phage (9.2 vs. 9.5×10¹⁰ ml/min; SΔ1/wild-type=0.97).

### EXAMPLE 9

Primers cysF (CTATTAACGGACTTTTGAGA, SEQ ID NO.: 7) and cysR (TTCAATACGTCCAATAGTTT SEQ ID NO.: 8) amplify the central rod region of phage T4 gene 37 including the location of the SΔ1 deletion and we used them to screen pseudorevertant phage as well as for sequencing. These primers amplify a 1.4-kb fragment from wild-type T4 DNA but only a 0.36-kb product from T4 37SΔ1 DNA. Primers recF (GACGAGCTCCTTCGGGTTCCCTTTTTCTTTA, SEQ ID NO.: 9) and 37B-2R (TTGGGTAACTCGACATGA, SEQ ID NO.: 10) amplify a 3.2-kb segment of the tail fiber gene cluster including the 3′ end of gene 35, gene 36, and the first two-thirds of gene 37. When these primers are used to amplify T4 37SΔ1, a 2.1-kb fragment is produced in which the deletion junction is approximately in the middle. We cloned this 2.1-kb PCR product into pGEM-T (Promega) for sequencing, further modification (see below), and to transfer modified genes into T4 phage by recombination between the plasmid and infecting phage. The construct containing this 2.1-kb insert was designated p37SΔ1.

### EXAMPLE 10

We transferred modified genes into phage by infecting plasmid bearing cells with T4 37amA481 (whose amber mutation is located in the segment of DNA that is missing in T4 37SΔ1 and its derivatives) and growing the phage to produce a stock. Because MC1061 is not an amber-suppressing strain, only cells where recombination between the plasmid and phage genome occurred would produce viable pseudorevertant phage. We selected recombinant phage from the lysates by plating on BB (su⁰) and screened, plaques by PCR to identify which plaques contained the 37SΔ1 deletion.

### EXAMPLE 11

In the β-sheets forming the central rod regions of the tail fibers, the loop regions contribute little to maintaining the H-bond network, nor to the van der Waals interaction in the hydrophobic layer within the rod. (Branden, C. & Tooze, J. (1999) Introduction to Protein Structure (Garland, N.Y.); Xu, G., Wang, W., Groves, J. T. & Hecht, M. H. (2001) Proc. Natl. Acad. Sci. USA 98, 3652-3657.) We postulate that the loops can be more variable and flexible than other regions of the tail fiber proteins. This postulate suggests that the junction of the SΔ1 deletion is in a loop (rather than in a-strand) of the rod portion of gene 37. Surface loops in proteins can often be expanded to include additional peptide sequences with minimal effects on protein structure, function or stability. (Regan, L. (1999) Curr. Opin. Struct. Biol. 9, 494-499.) Thus, if the SΔ1 junction is in a loop, we should be able to insert additional sequences into the junction, expanding the loop, without severely disrupting the structural integrity of the tail fiber.

To test this we added DNA sequences encoding a pentaglycine peptide into the SΔ1 junction (Table 1) in the cloned gene segment. The pentaglycine coding segment in SΔ1G5 was added to the cloned DNA in p37SΔ1/T by using overlapping PCR primers. Primers 37SΔ1-1F (GGCGATGGTGGCGGTGGCGGCAATGTACAATTTTACGCTG, SEQ ID NO.: 11) and 37SΔ1-1R (TACATTGCCGCCACCGCCACCATCGCCATTTAATCTCAA, SEQ ID NO.: 12) contain complementary sequences corresponding to the Gly-5-containing SΔ1 junction. They were used with the flanking recF and 37B-2R primers to produce two modified half segments that were then recombined using the complementary ends to fuse the,two segments and the flanking primers to amplify the whole segment. The entire segment was then cloned into pGEM-T.

SΔ1UCS (universal cloning site) was creating by amplifying half segments of the SΔ1 clone with primers 37SΔ1-2F GGCGATGAGACGGTACCGTCTCAATGTACAATTTTACGCTG, SEQ ID NO.: 13 and 37SΔ1-2R TACATTGAGACGGTACCGTCTCATCGCCATTTAATCTCAA, SEQ ID NO.: 14. Each primer contains a BsmBI and KpnI site. The two half segments were joined using the KpnI site to create a single segment with two BsmBI sites around the central KpnI site inserted into the SΔ1 junction. BsmBI cuts at positions 7/11 outside of the recognition site and the two BsmBI sites in p37SΔ1UCS/T are arranged so that the two cuts drop out the center segment (containing both BsmBI sites) leaving the original construct sequence with two different cohesive ends. This arrangement allows for the insertion (with an unambiguous orientation) of any double-stranded oligonucleotide with the correct cohesive ends. Thus, any oligopeptide can be cloned into junction of the SΔ1 deletion. We inserted the raSΔ1 control (nonepitopic for monoclonal antibody Y13-259) sequence by combining the oligonucleotides SΔ1R-1F GCGATGGTGGCGGTGGCGCCCGCGGCGTGGGAAAGAGTGCCCTGACCATCCAGCTG ATCGGTGGCGGTGGCA, SEQ ID NO.: 15 and SΔ1R-1R ACATTGCCACCGCCACCGATCAGCTGGATGGTCAGGGCACTCTTTCCCACGCCGCGG GCGCCACCGCCACCA, SEQ ID NO.: 16 Similarly, the ras2 mAb epitope coding sequence was inserted by using the oligonucleotides SΔ1R-2F GCGATGGTGGCGGTGGCGAAGAATACTCCGCAATGCGCGACCAGTACATGCGCACC GGTGAAGGTGGCGGTGGCA, SEQ ID NO.: 17 and SΔ1R-2R ACATTGCCACCGCCACCTTCACCGGTGCGCATGTACTGGTCGCGCATTGCGGAGTAT TCTTCGCCACCGCCACCA, SEQ ID NO.: 18. To anneal each oligonucleotide pair, we mixed the appropriate oligonucleotides in equimolar amounts, boiled the mixtures briefly, and cooled the mixtures slowly to form the appropriate double-stranded oligonucleotides with the correct single-stranded extensions. These oligonucleotides were ligated directly into BsmBI-digested p37SΔ1UCS/T. The insertions were confirmed by sequencing with the cysF primer.

This modified sequence also transferred readily into phage by homologous recombination. The SΔ1G5 phage produced poorer stocks, although the adsorption constant was almost the same as for the wild-type and SΔ1 phage (12×10¹⁰ ml/min; SΔ1G5/wild type=1.3). Poorer stocks might indicate a mild interference with phage development. This finding confirms that the SΔ1 junction is able to accept peptide insertions without any significant loss of structural integrity and fits our hypothesis that the junction identifies a loop in the β-structure which can be used as an insertion point for functional moieties in the structural unit.

### EXAMPLE 12

To use tail fiber derived proteins as mesoscale assembly units, we may also need to attach specific functions to the assembled arrays of structural units. They may be attached before or after maturation of the final structure or at an intermediate step. The attachment may be covalent (e.g., disulfide bridges) or noncovalent (e.g., his tags). Incorporation of a peptide epitope may also be used to attach a functionality linked to the appropriate antibody. Fusions between antibodies and functional peptides have been extensively developed. (Vitetta, E. S., Fulton, R. J., May, R. D. , Till, M. & Uhr, J. W. (1987) Science 238, 1098-1104; Byers, V. S. & Baldwin, R. W. (1988) Immunology 65, 329-335.) In the case of our nanoarchitectures, the compound would be fused to a mAb that is specific for an epitope in the structural unit. Thus, we set out to show that an antibody epitope could be incorporated into a tail fiber protein.

To do this, we inserted two different 15 aa sequences from the human H-ras gene into the putative loop at the SΔ1 fusion junction (Table 1). Both peptides were flanked by four glycines on each side. One construct, SΔ1ras1, containing a nonepitope segment of H-ras, was created as a control, whereas the other, SΔ1ras2, contains the epitope specifically recognized by the rat monoclonal IgG antibody Y13-259. (Sigal, I. S., Gibbs, J. B., D'Alonzo, J. S. & Scolnick, E. M. (1986) Proc. Natl. Acad. Sci. USA 83, 4725-4729.) Each of these modified genes readily transferred into phage by homologous recombination.

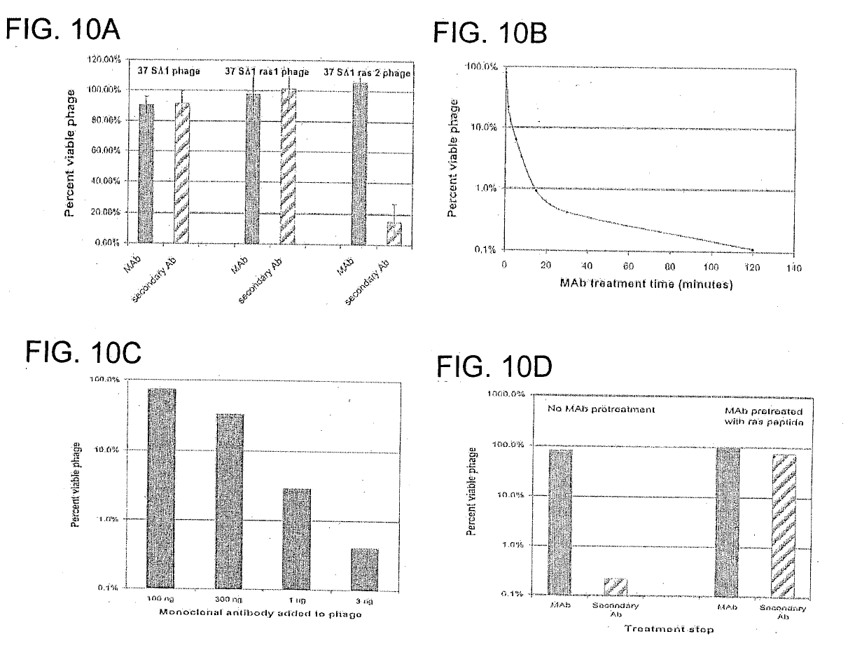

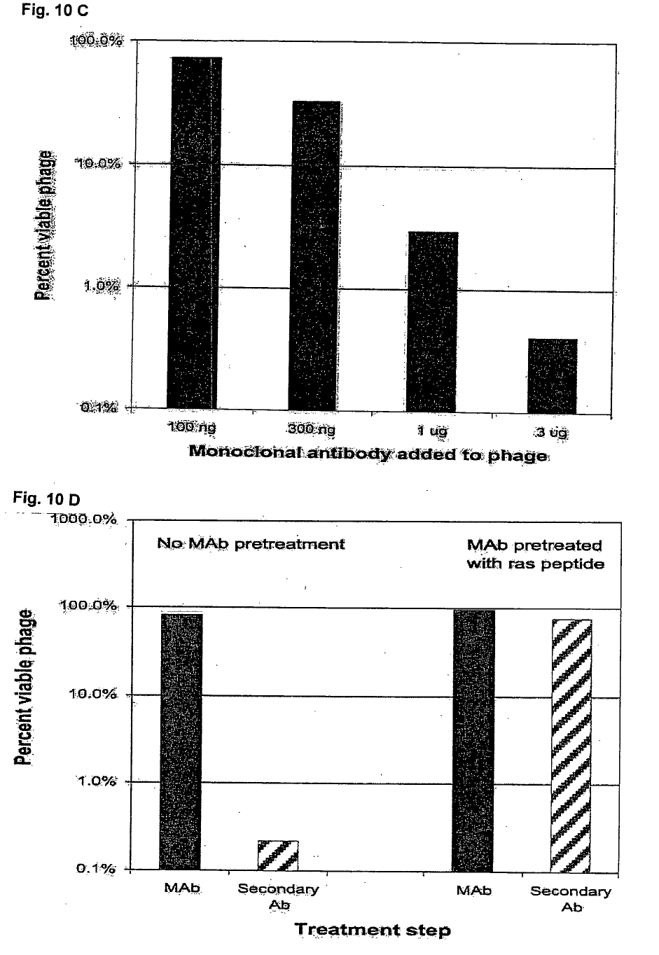

To test whether the epitope was accessible for interactions with the exogenous antibody, we treated SΔ1, SΔ1raSΔ1, and SΔ1ras2 phage with the anti-ras mAb. We purchased mAb Ab-1 Y13-259 and inactivating peptide from Calbiochem and rabbit anti-rat whole IgG serum from Sigma. The mAb and peptide were resuspended in Dulbecco's PBS and the anti-serum was used as supplied. For inactivation experiments, we diluted phage to 10¹⁰ cells/ml in 10 mM phosphate pH 7.4/10 mM MgSO₄. We added mAb (from a 0.1 mg/ml stock) to 500 μl of diluted phage and incubated the mixture for 30 min (unless otherwise indicated) at room temperature on a rotisserie mixer. Then we added 4 μg of secondary antiserum (from a 2 mg/ml stock) and incubated for 30 min at room temperature. For the initial experiments shown in FIG. 10A we used 1 μg of mAb, whereas 3 μg or the indicated amount was used for the remaining experiments. For the free epitope inhibition experiment shown in FIG. 10D, we mixed the peptide (EEYSAMRDQVMRTGE, SEQ ID NO.: 19) and mAb at a 10:1 molar ratio and incubated for 30 min at room temperature. The mAb/peptide mixture was then added to phage as described above.

If the mAb can bind to the H-ras epitope, it might inactivate the phage by linking together tail fibers on a single phage, thereby preventing proper binding to the cell surface. Alternatively, several phage might be linked together to form large noninfectious complexes. However, as FIG. 10A shows, mAb treatment alone (gray bars) did not result in phage inactivation. When the phage/mAb mixtures were further treated with an anti-rat IgG serum (striped bars) (which binds to the Fc region of the mAb), 85% of the SΔ1ras2 phage were inactivated. Because the SΔ1ras1 control phage were unaffected and because the anti-rat IgG antiserum alone has no effect on the SΔ1ras2 phage, this finding demonstrates that the ras2 epitope is exposed on the surface of the tail fiber and accessible to the mAb. The requirement for the secondary antibody for phage inactivation may reflect the axial symmetry of P37. Because each mature fiber contains more than one epitope in close proximity on each tail fiber, it is likely that both binding sites in the mAb become bound to a single fiber. This would not be expected to inactivate the phage. Hence, the need for the secondary antibodies to crosslink tail fibers by binding to two mAbs bound to two different fibers and inactivate the phage. Regardless of the specific mechanism of inactivation, these experiments show that a functional peptide can be added to the rod region of a tail fiber protein without disrupting the tail fiber structure or function.

We further investigated the interaction of the SΔ1ras2 phage with the mAb. FIG. 10B shows that inactivation depends on the time allowed for mAb binding before addition of the secondary antiserum, reaching a maximum of 99.9% by 120 min. FIG. 10C shows that inactivation also has a simple dose-response relationship with the amount of Y13-259 mAb used. FIG. 10D shows that the SΔ1ras2 phage could be protected from inactivation by pretreating the mAb with a free 15-aa peptide of the same sequence as the 15-residue epitope inserted into the tail fiber protein. Although 99.8% of the phage were inactivated in the control treatment (with buffer only), there was no significant inactivation when the mAb was pretreated with the peptide. This finding demonstrates that the inactivation requires a specific interaction of the antibody with its specific epitope sequence.

We examined how the mAb interacts with the tail fiber by imaging mAb-treated phage, and observed a typical cluster of inactivated phage. The phage form a “bouquet” with the tail fibers linked together. It is unlikely that phage in such a bouquet could orient properly on the cell surface to allow the tail fibers to function cooperatively and trigger infection.

### EXAMPLE 13

Phage and phage/antibody complexes were stained with 1% phosphotungstate (pH 7) on carbon grids. Grids were examined at 100 kV by using a Philips CM10 transmission electron microscope. The final micrograph images were at a magnification of ×73,000.

Phage carrying the SΔ1 mutation was observed to have a shortened distal portion of the tail fiber. To compare wild-type tail fiber to SΔ1 tail fiber we calculated the ratio of the lengths of the distal half fiber/proximal half fiber (D/P) by using measurements from enlarged electron micrographs. We found that for wild-type fibers D/P=0.99±0.06 (n=11) and for SΔ1 fibers D/P=0.54±0.14 (n=6). This finding confirms that the viable SΔ1 phage have shortened but otherwise functional tail fibers.

TABLE 1
Partial protein sequences of naturally occurring and engineered gene 37
proteins Phage Partial protein sequence of gene 37 at SΔ1 junction

| | | |
|---|---|---|
| Wild-type T4 | GLLRLNGDYVQ//GSNNVQFYADG | SEQ ID NO.:20 |
| 37 SΔ1 | GLLRLNGD\|NVQFYADG | SEQ ID NO.:21 |
| 37 SΔ1G5 | GLLRLNGDGGGGGNVQFYADG | SEQ ID NO.:22 |
| 37 SΔ1ras1 (control) | GLLRLNGDGGGGARGVGKSALTIQLIGGGGNVQFYADG | SEQ ID NO.:23 |
| 37 SΔ1ras2 (mAb epitope) | GLLRLNGDGGGGEEYSAMRDQYMRTGEGGGGNVQFYADG | SEQ ID NO.:24 |

Sequences flanking the SΔ1 junction are in italics, double slash represents 340 deleted amino acid residues, vertical line marks the position of the junction, inserted sequences are in boldface.

1. A method for making a nanostructure comprising a plurality of structural units in which the positions of the structural units relative to each other are established in a defined geometry, comprising the step of sequentially adding structural units to a growing structure to build up the nanostructure,
wherein the nanostructure comprises two or more species of protein structural units;
wherein each structural unit is added to the growing structure in a separate structural unit-addition step and structural unit nots incorporated in the growing structure are removed at the end of each structural unit-addition step; and
wherein each species of structural unit has the ability to assemble non-covalently with the growing nanostructure to which it is added but cannot self-assemble with other structural units of the same species.

2. The method of claim 1, wherein the nanostructure is a two or three dimensional structure.

3. The method of claim 2, wherein the structural units are added to a growing structure that is immobilized on a support.

4. The method of claim 3, further comprising the step of releasing the nanostructure from the solid support after addition of the structural units to the growing structure.

5. The method according to claim 2, wherein at least one of the structural units is a protein structural unit in the form of a stiff rod.

6. The method according to claim 5, wherein the protein structural unit in the form of a stiff rod are derived from T-even bacteriophage tail fibers.

7. The method of claim 6, wherein the protein structural units in the form of stiff rods are derived from bacteriophage T4 gp34, gp35, gp36 or gp37 tail fibers.

8. The method of claim 2, wherein at least one of the structural units is a chimeric protein.

9. The method of claim 8, wherein the chimeric protein contains sequences from two or more different bacteriophage T-even tail fiber proteins.

10. The method of claim 8, wherein the fusion protein contains sequences from a bacteriophage T-even tail fiber protein and another protein or peptide.

11. The method of claim 10, wherein the other protein or peptide comprises an epitope recognized by an antibody.

12. The method of claim 1, wherein the structural units are added to a growing structure that is immobilized on a support.

13. The method of claim 12, further comprising the step of releasing the nanostructure from the solid support after addition of the structural units to the growing structure.

14. The method according to claim 12, wherein at least one of the structural units is a protein structural unit in the form of a stiff rod.

15. The method according to claim 14, wherein the protein structural unit in the form of a stiff rods are derived from T-even bacteriophage tail fibers.

16. The method of claim 15, wherein the protein structural units in the form of stiff rods are derived from bacteriophage T4 gp34, gp35, gp36 or gp37 tail fibers.

17. The method of claim 12, wherein at least one of the structural units is a chimeric protein.

18. The method of claim 17, wherein the chimeric protein contains sequences from two or more different bacteriophage T-even tail fiber proteins.

19. The method of claim 17, wherein the fusion protein contains sequences from a bacteriophage T-even tail fiber protein and another protein or peptide.

20. The method of claim 19, wherein the other protein or peptide comprises an epitope recognized by an antibody.

21. The method according to claim 1, wherein at least one of the structural units is a protein structural unit in the form of a stiff rods.

22. The method according to claim 21, wherein the protein structural unit in the form of a stiff rods are derived from T-even bacteriophage tail fibers.

23. The method of claim 22, wherein the protein structural units in the form of stiff rods are derived from bacteriophage T4 gp34, gp35, gp36 or gp37 tail fibers.

24. The method of claim 1, wherein at least one of the structural units is a chimeric protein.

25. The method of claim 24, wherein the chimeric protein contains sequences from two or more different bacteriophage T-even tail fiber proteins.

26. The method of claim 24, wherein the fusion protein contains sequences from a bacteriophage T-even tail fiber protein and another protein or peptide.

27. The method of claim 26, wherein the other protein or peptide comprises an epitope recognized by an antibody.

28. The method of claim 1, wherein at least a portion of a nanostructure comprises a first structural subunit A, having binding sites A1 and A2 and a second structural subunit B, having binding sites B1 and B2, wherein A2 binds to B1 and A1 binds to B2, arranged in an alternating sequence.

29. The method of claim 28, wherein the nanostructure is a two or three dimensional structure.

30. The method of claim 29, wherein the structural units are added to a growing structure that is immobilized on a support.

31. The method of claim 28, further comprising the step of releasing the nanostructure from the solid support after addition of the structural units to the growing structure.

32. The method according to claim 28, wherein at least one of the structural units is a protein structural unit in the form of a stiff rods.

33. The method according to claim 32, wherein the protein structural unit in the form of a stiff rods are derived from T-even bacteriophage tail fibers.

34. The method of claim 33 wherein the protein structural units in the form of stiff rods are derived from bacteriophage T4 gp34, gp35, gp36 or gp37 tail fibers.

35. The method of claim 28, wherein at least one of the structural units is a chimeric protein.

36. The method of claim 35, wherein the chimeric protein contains sequences from two or more different bacteriophage T-even tail fiber proteins.

37. The method of claim 35, wherein the fusion protein contains sequences from a bacteriophage T-even tail fiber protein and another protein or peptide.

38. The method of claim 37, wherein the other protein or peptide comprises an epitope recognized by an antibody.

39. The method of claim 28, wherein the structural units are added to a growing structure that is immobilized on a support.

40. The method of claim 39, further comprising the step of releasing the nanostructure from the solid support after addition of the structural units to the growing structure.


SEQUENCE LISTING (24 sequences)

SEQ ID NO: 1 - DNA, 8855 bp, Bacteriophage T4
taggagcccgggagaatggccgagattaaaagagaattcagagcagaagatggtctggacgcaggtggtgataaaataatcaacgtagctttagctgatcgtaccgtaggaactgacggtgttaacgttgattacttaattcaagaaaacacagttcaacagtatgatccaactcgtggatatttaaaagattttgtaatcatttatgataaccgcttttgggctgctataaatgatattccaaaaccagcaggagcttttaatagcggacgctggagagcattacgtaccgatgctaactggattacggtttcatctggttcatatcaattaaaatctggtgaagcaatttcggttaacaccgcagctggaaatgacatcacgtttactttaccatcttctccaattgatggtgatactatcgttctccaagatattggaggaaaacctggagttaaccaagttttaattgtagctccagtacaaagtattgtaaactttagaggtgaacaggtacgttcagtactaatgactcatccaaagtcacagctagttttaatttttagtaatcgtctgtggcaaatgtatgttgctgattatagtagagaagctatagttgtaacaccagcgaatacttatcaagcgcaatccaacgattttatcgtacgtagatttacttctgctgcaccaattaatgtcaaacttccaagatttgctaatcatggcgatattattaatttcgtcgatttagataaactaaatccgctttatcatacaattgttactacatacgatgaaacgacttcagtacaagaagttggaactcattccattgaaggccgtacatcgattgacggtttcttgatgtttgatgataatgagaaattatggagactgtttgacggggatagtaaagcgcgtttacgtatcataacgactaattcaaacattcgtccaaatgaagaagttatggtatttggtgcgaataacggaacaactcaaacaattgagcttaagcttccaactaatatttctgttggtgatactgttaaaatttccatgaattacatgagaaaaggacaaacagttaaaatcaaagctgctgatgaagataaaattgcttcttcagttcaattgctgcaattcccaaaacgctcagaatatccacctgaagctgaatgggttacagttcaagaattagtttttaacgatgaaactaattatgttccagttttggagcttgcttacatagaagattctgatggaaaatattgggttgtacagcaaaacgttccaactgtagaaagagtagattctttaaatgattctactagagcaagattaggcgtaattgctttagctacacaagctcaagctaatgtcgatttagaaaattctccacaaaaagaattagcaattactccagaaacgttagctaatcgtactgctacagaaactcgcagaggtattgcaagaatagcaactactgctcaagtgaatcagaacaccacattctcttttgctgatgatattatcatcactcctaaaaagctgaatgaaagaactgctacagaaactcgtagaggtgtcgcagaaattgctacgcagcaagaaactaatgcaggaaccgatgatactacaatcatcactcctaaaaagcttcaagctcgtcaaggttctgaatcattatctggtattgtaacctttgtatctactgcaggtgctactccagcttctagccgtgaattaaatggtacgaatgtttataataaaaacactgataatttagttgtttcacctaaagctttggatcagtataaagctactccaacacagcaaggtgcagtaattttagcagttgaaagtgaagtaattgctggacaaagtcagcaaggatgggcaaatgctgttgtaacgccagaaacgttacataaaaagacatcaactgatggaagaattggtttaattgaaattgctacgcaaagtgaagttaatacaggaactgattatactcgtgcagtcactcctaaaactttaaatgaccgtagagcaactgaaagtttaagtggtatagctgaaattgctacacaagttgaattcgacgcaggcgtcgacgatactcgtatctctacaccattaaaaattaaaaccagatttaatagtactgatcgtacttctgttgttgctctatctggattagttgaatcaggaactctctgggaccattatacacttaatattcttgaagcaaatgagacacaacgtggtacacttcgtgtagctacgcaggtcgaagctgctgcgggaacattagataatgttttaataactcctaaaaagcttttaggtactaaatctactgaagcgcaagagggtgttattaaagttgcaactcagtctgaaactgtgactggaacgtcagcaaatactgctgtatctccaaaaaatttaaaatggattgcgcagagtgaacctacttgggcagctactactgcaataagaggttttgttaaaacttcatctggttcaattacattcgttggtaatgatacagtcggttctacccaagatttagaactgtatgagaaaaatagctatgcggtatcaccatatgaattaaaccgtgtattagcaaattatttgccactaaaagcaaaagctgctgatacaaatttattggatggtctagattcatctcagttcattcgtagggatattgcacagacggttaatggttcactaaccttaacccaacaaacgaatctgagtgcccctcttgtatcatctagtactggtgaatttggtggttcattggccgctaatagaacatttaccatccgtaatacaggagccccgactagtatcgttttcgaaaaaggtcctgcatccggggcaaatcctgcacagtcaatgagtattcgtgtatggggtaaccaatttggcggcggtagtgatacgacccgttcgacagtgtttgaagttggcgatgacacatctcatcacttttattctcaacgtaataaagacggtaatatagcgtttaacattaatggtactgtaatgccaataaacattaatgcttccggtttgatgaatgtgaatggcactgcaacattcggtcgttcagttacagccaatggtgaattcatcagcaagtctgcaaatgcttttagagcaataaacggtgattacggattctttattcgtaatgatgcctctaatacctattttttgctcactgcagccggtgatcagactggtggttttaatggattacgcccattattaattaataatcaatccggtcagattacaattggtgaaggcttaatcattgccaaaggtgttactataaattcaggcggtttaactgttaactcgagaattcgttctcagggtactaaaacatctgatttatatacccgtgcgccaacatctgatactgtaggattctggtcaatcgatattaatgattcagccacttataaccagttcccgggttattttaaaatggttgaaaaaactaatgaagtgactgggcttccatacttagaacgtggcgaagaagttaaatctcctggtacactgactcagtttggtaacacacttgattcgctttaccaagattggattacttatccaacgacgccagaagcgcgtaccactcgctggacacgtacatggcagaaaaccaaaaactcttggtcaagttttgttcaggtatttgacggaggtaaccctcctcaaccatctgatatcggtgctttaccatctgataatgctacaatggggaatcttactattcgtgatttcttgcgaattggtaatgttcgcattgttcctgacccagtgaataaaacggttaaatttgaatgggttgaataagaggtattatggaaaaatttatggccgagatttggacaaggatatgtccaaacgccattttatcggaaagtaattcagtaagatataaaataagtatagcgggttcttgcccgctttctacagcaggaccatcatatgttaaatttcaggataatcctgtaggaagtcaaacatttaggcgcaggccttcatttaagagtttttgacccttccaccggagcattagttgatagtaagtcatatgctttttcgacttcaaatgatactacatcagctgcttttgttagttttcatgaattctttgacgaataatcgaattgttgctatattaactagtggaaaggttaattttcctcctgaagtagtatcttggttaagaaccgccggaacgtctgcctttccatctgattctatattgtcaagatttgacgtatcatatgctgctttttatacttcttctaaaagagctatcgcattagagcatgttaaactgagtaatagaaaaagcacagatgattatcaaactattttagatgttgtatttgacagtttagaagatgtaggagctaccgggtttccaagaagaacgtatgaaagtgttgagcaattcatgtcggcagttggtggaactaataacgaaattgcgagattgccaacttcagctgctataagtaaattatctgattataatttaattcctggagatgttctttatcttaaagctcagttatatgctgatgctgatttacttgctcttggaactacaaatatatctatccgtttttataatgcatctaacggatatatttcttcaacacaagctgaatttactgggcaagctgggtcatgggaattaaaggaagattatgtagttgttccagaaaacgcagtaggatttacgatatacgcacagagaactgcacaagctggccaaggtggcatgagaaatttaagcttttctgaagtatcaagaaatggcggcatttcgaaacctgctgaatttggcgtcaatggtattcgtgttaattatatctgcgaatccgcttcacctccggatataatggtacttcctacgcaagcatcgtctaaaactggtaaagtgtttgggcaagaatttagagaagtttaaattgagggacccttcgggttccctttttctttataaatactattcaaataaaggggcatacaatggctgatttaaaagtaggttcaacaactggaggctctgtcatttggcatcaaggaaattttccattgaatccagccggtgacgatgtactctataaatcatttaaaatatattcagaatataacaaaccacaagctgctgataacgatttcgtttctaaagctaatggtggtacttatgcatcaaaggtaacatttaacgctggcattcaagtcccatatgctccaaacatcatgagcccatgcgggatttatgggggtaacggtgatggtgctacttttgataaagcaaatatcgatattgtttcatggtatggcgtaggatttaaatcgtcatttggttcaacaggccgaactgttgtaattaatacacgcaatggtgatattaacacaaaaggtgttgtgtcggcagctggtcaagtaagaagtggtgcggctgctcctatagcagcgaatgaccttactagaaaggactatgttgatggagcaataaatactgttactgcaaatgcaaactctagggtgctacggtctggtgacaccatgacaggtaatttaacagcgccaaactttttctcgcagaatcctgcatctcaaccctcacacgttccacgatttgaccaaatcgtaattaaggattctgttcaagatttcggctattattaagaggacttatggctactttaaaacaaatacaatttaaaagaagcaaaatcgcaggaacacgtcctgctgcttcagtattagccgaaggtgaattggctataaacttaaaagatagaacaatttttactaaagatgattcaggaaatatcatcgatctaggttttgctaaaggcgggcaagttgatggcaacgttactattaacggacttttgagattaaatggcgattatgtacaaacaggtggaatgactgtaaacggacccattggttctactgatggcgtcactggaaaaattttcagatctacacagggttcattttatgcaagagcaacaaacgatacttcaaatgcccatttatggtttgaaaatgccgatggcactgaacgtggcgttatatatgctcgccctcaaactacaactgacggtgaaatacgccttagggttagacaaggaacaggaagcactgccaacagtgaattctatttccgctctataaatggaggcgaatttcaggctaaccgtattttagcatcagattcgttagtaacaaaacgcattgcggttgataccgttattcatgatgccaaagcatttggacaatatgattctcactctttggttaattatgtttatcctggaaccggtgaaacaaatggtgtaaactatcttcgtaaagttcgcgctaagtccggtggtacaatttatcatgaaattgttactgcacaaacaggcctggctgatgaagtttcttggtggtctggtgatacaccagtatttaaactatacggtattcgtgacgatggcagaatgattatccgtaatagccttgcattaggtacattcactacaaatttcccgtctagtgattatggcaacgtcggtgtaatgggcgataagtatcttgttctcggcgacactgtaactggcttgtcatacaaaaaaactggtgtatttgatctagttggcggtggatattctgttgcttctattactcctgacagtttccgtagtactcgtaaaggtatatttggtcgttctgaggaccaaggcgcaacttggataatgcctggtacaaatgctgctctcttgtctgttcaaacacaagctgataataacaatgctggagacggacaaacccatatcgggtacaatgctggcggtaaaatgaaccactatttccgtggtacaggtcagatgaatatcaatacccaacaaggtatggaaattaacccgggtattttgaaattggtaactggctctaataatgtacaattttacgctgacggaactatttcttccattcaacctattaaattagataacgagatatttttaactaaatctaataatactgcgggtcttaaatttggagctcctagccaagttgatggcacaaggactatccaatggaacggtggtactcgcgaaggacagaataaaaactatgtgattattaaagcatggggtaactcatttaatgccactggtgatagatctcgcgaaacggttttccaagtatcagatagtcaaggatattatttttatgctcatcgtaaagctccaaccggcgacgaaactattggacgtattgaagctcaatttgctggggatgtttatgctaaaggtattattgccaacggaaattttagagttgttgggtcaagcgctttagccggcaatgttactatgtctaacggtttgtttgtccaaggtggttcttctattactggacaagttaaaattggcggaacagcaaacgcactgagaatttggaacgctgaatatggtgctattttccgtcgttcggaaagtaacttttatattattccaaccaatcaaaatgaaggagaaagtggagacattcacagctctttgagacctgtgagaataggattaaacgatggcatggttgggttaggaagagattcttttatagtagatcaaaataatgctttaactacgataaacagtaactctcgcattaatgccaactttagaatgcaattggggcagtcggcatacattgatgcagaatgtactgatgctgttcgcccggcgggtgcaggttcatttgcttcccagaataatgaagacgtccgtgcgccgttctatatgaatattgatagaactgatgctagtgcatatgttcctattttgaaacaacgttatgttcaaggcaatggctgctattcattagggactttaattaataatggtaatttccgagttcattaccatggcggcggagataacggttctacaggtccacagactgctgattttggatgggaatttattaaaaacggtgattttatttcacctcgcgatttaatagcaggcaaagtcagatttgatagaactggtaatatcactggtggttctggtaattttgctaacttaaacagtacaattgaatcacttaaaactgatatcatgtcgagttacccaattggtgctccgattccttggccgagtgattcagttcctgctggatttgctttgatggaaggtcagacctttgataagtccgcatatccaaagttagctgttgcatatcctagcggtgttattccagatatgcgcgggcaaactatcaagggtaaaccaagtggtcgtgctgttttgagcgctgaggcagatggtgttaaggctcatagccatagtgcatcggcttcaagtactgacttaggtactaaaaccacatcaagctttgactatggtacgaagggaactaacagtacgggtggacacactcactctggtagtggttctactagcacaaatggtgagcacagccactacatcgaggcatggaatggtactggtgtaggtggtaataagatgtcatcatatgccatatcatacagggcgggtgggagtaacactaatgcagcagggaaccacagtcacactttctcttttgggactagcagtgctggcgaccattcccactctgtaggtattggtgctcatacccacacggtagcaattggatcacatggtcatactatcactgtaaatagtacaggtaatacagaaaacacggttaaaaacattgcttttaactatatcgttcgtttagcataaggagaggggcttcggcccttctaa

SEQ ID NO: 2 - protein, 1289 aa, Bacteriophage T4
MAEIKREFRAEDGLDAGGDKIINVALADRTVGTDGVNVDYLIQENTVQQYDPTRGYLKDFVIIYDNRFWAAINDIPKPAGAFNSGRWRALRTDANWITVSSGSYQLKSGEAISVNTAAGNDITFTLPSSPIDGDTIVLQDIGGKPGVNQVLIVAPVQSIVNFRGEQVRSVLMTHPKSQLVLIFSNRLWQMYVADYSREAIVVTPANTYQAQSNDFIVRRFTSAAPINVKLPRFANHGDIINFVDLDKLNPLYHTIVTTYDETTSVQEVGTHSIEGRTSIDGFLMFDDNEKLWRLFDGDSKARLRIITTNSNIRPNEEVMVFGANNGTTQTIELKLPTNISVGDTVKISMNYMRKGQTVKIKAADEDKIASSVQLLQFPKRSEYPPEAEWVTVQELVFNDETNYVPVLELAYIEDSDGKYWVVQQNVPTVERVDSLNDSTRARLGVIALATQAQANVDLENSPQKELAITPETLANRTATETRRGIARIATTAQVNQNTTFSFADDIIITPKKLNERTATETRRGVAEIATQQETNAGTDDTTIITPKKLQARQGSESLSGIVTFVSTAGATPASSRELNGTNVYNKNTDNLVVSPKALDQYKATPTQQGAVILAVESEVIAGQSQQGWANAVVTPETLHKKTSTDGRIGLIEIATQSEVNTGTDYTRAVTPKTLNDRRATESLSGIAEIATQVEFDAGVDDTRISTPLKIKTRFNSTDRTSVVALSGLVESGTLWDHYTLNILEANETQRGTLRVATQVEAAAGTLDNVLITPKKLLGTKSTEAQEGVIKVATQSETVTGTSANTAVSPKNLKWIAQSEPTWAATTAIRGFVKTSSGSITFVGNDTVGSTQDLELYEKNSYAVSPYELNRVLANYLPLKAKAADTNLLDGLDSSQFIRRDIAQTVNGSLTLTQQTNLSAPLVSSSTGEFGGSLAANRTFTIRNTGAPTSIVFEKGPASGANPAQSMSIRVWGNQFGGGSDTTRSTVFEVGDDTSHHFYSQRNKDGNIAFNINGTVMPININASGLMNVNGTATFGRSVTANGEFISKSANAFRAINGDYGFFIRNDASNTYFLLTAAGDQTGGFNGLRPLLINNQSGQITIGEGLIIAKGVTINSGGLTVNSRIRSQGTKTSDLYTRAPTSDTVGFWSIDINDSATYNQFPGYFKMVEKTNEVTGLPYLERGEEVKSPGTLTQFGNTLDSLYQDWITYPTTPEARTTRWTRTWQKTKNSWSSFVQVFDGGNPPQPSDIGALPSDNATMGNLTIRDFLRIGNVRIVPDPVNKTVKFEWVE

SEQ ID NO: 3 - protein, 65 aa, Bacteriophage T4
MEKFMAEIWTRICPNAILSESNSVRYKISIAGSCPLSTAGPSYVKFQDNPVGSQTFRRRPSFKSF

SEQ ID NO: 4 - protein, 295 aa, Bacteriophage T4
MLFRLQMILHQLLLLVFMNSLTNNRIVAILTSGKVNFPPEVVSWLRTAGTSAFPSDSILSRFDVSYAAFYTSSKRAIALEHVKLSNRKSTDDYQTILDVVFDSLEDVGATGFPRRTYESVEQFMSAVGGTNNEIARLPTSAAISKLSDYNLIPGDVLYLKAQLYADADLLALGTTNISIRFYNASNGYISSTQAEFTGQAGSWELKEDYVVVPENAVGFTIYAQRTAQAGQGGMRNLSFSEVSRNGGISKPAEFGVNGIRVNYICESASPPDIMVLPTQASSKTGKVFGQEFREV

SEQ ID NO: 5 - protein, 221 aa, Bacteriophage T4
MADLKVGSTTGGSVIWHQGNFPLNPAGDDVLYKSFKIYSEYNKPQAADNDFVSKANGGTYASKVTFNAGIQVPYAPNIMSPCGIYGGNGDGATFDKANIDIVSWYGVGFKSSFGSTGRTVVINTRNGDINTKGVVSAAGQVRSGAAAPIAANDLTRKDYVDGAINTVTANANSRVLRSGDTMTGNLTAPNFFSQNPASQPSHVPRFDQIVIKDSVQDFGYY

SEQ ID NO: 6 - protein, 1026 aa, Bacteriophage T4
MATLKQIQFKRSKIAGTRPAASVLAEGELAINLKDRTIFTKDDSGNIIDLGFAKGGQVDGNVTINGLLRLNGDYVQTGGMTVNGPIGSTDGVTGKIFRSTQGSFYARATNDTSNAHLWFENADGTERGVIYARPQTTTDGEIRLRVRQGTGSTANSEFYFRSINGGEFQANRILASDSLVTKRIAVDTVIHDAKAFGQYDSHSLVNYVYPGTGETNGVNYLRKVRAKSGGTIYHEIVTAQTGLADEVSWWSGDTPVFKLYGIRDDGRMIIRNSLALGTFTTNFPSSDYGNVGVMGDKYLVLGDTVTGLSYKKTGVFDLVGGGYSVASITPDSFRSTRKGIFGRSEDQGATWIMPGTNAALLSVQTQADNNNAGDGQTHIGYNAGGKMNHYFRGTGQMNINTQQGMEINPGILKLVTGSNNVQFYADGTISSIQPIKLDNEIFLTKSNNTAGLKFGAPSQVDGTRTIQWNGGTREGQNKNYVIIKAWGNSFNATGDRSRETVFQVSDSQGYYFYAHRKAPTGDETIGRIEAQFAGDVYAKGIIANGNFRVVGSSALAGNVTMSNGLFVQGGSSITGQVKIGGTANALRIWNAEYGAIFRRSESNFYIIPTNQNEGESGDIHSSLRPVRIGLNDGMVGLGRDSFIVDQNNALTTINSNSRINANFRMQLGQSAYIDAECTDAVRPAGAGSFASQNNEDVRAPFYMNIDRTDASAYVPILKQRYVQGNGCYSLGTLINNGNFRVHYHGGGDNGSTGPQTADFGWEFIKNGDFISPRDLIAGKVRFDRTGNITGGSGNFANLNSTIESLKTDIMSSYPIGAPIPWPSDSVPAGFALMEGQTFDKSAYPKLAVAYPSGVIPDMRGQTIKGKPSGRAVLSAEADGVKAHSHSASASSTDLGTKTTSSFDYGTKGTNSTGGHTHSGSGSTSTNGEHSHYIEAWNGTGVGGNKMSSYAISYRAGGSNTNAAGNHSHTFSFGTSSAGDHSHSVGIGAHTHTVAIGSHGHTITVNSTGNTENTVKNIAFNYIVRLA

SEQ ID NO: 7 - DNA, 20 bp, Bacteriophage T4
ctattaacggacttttgaga

SEQ ID NO: 8 - DNA, 20 bp, Bacteriophage T4
ttcaatacgtccaatagttt

SEQ ID NO: 9 - DNA, 31 bp, Bacteriophage T4
gacgagctccttcgggttccctttttcttta

SEQ ID NO: 10 - DNA, 18 bp, Bacteriophage T4
ttgggtaactcgacatga

SEQ ID NO: 11 - DNA, 40 bp, Bacteriophage T4
ggcgatggtggcggtggcggcaatgtacaattttacgctg

SEQ ID NO: 12 - DNA, 39 bp, Bacteriophage T4
tacattgccgccaccgccaccatcgccatttaatctcaa

SEQ ID NO: 13 - DNA, 41 bp, artificial
  amplification primer 37S .delta. 1-2F
ggcgatgagacggtaccgtctcaatgtacaattttacgctg

SEQ ID NO: 14 - DNA, 40 bp, artificial
  amplification primer 37S .delta. 1-2R
tacattgagacggtaccgtctcatcgccatttaatctcaa

SEQ ID NO: 15 - DNA, 72 bp, artificial
  S .delta. 1R-1F
gcgatggtggcggtggcgcccgcggcgtgggaaagagtgccctgaccatccagctgatcggtggcggtggca

SEQ ID NO: 16 - DNA, 72 bp, artificial
  S .delta. 1R-1R
acattgccaccgccaccgatcagctggatggtcagggcactctttcccacgccgcgggcgccaccgccacca

SEQ ID NO: 17 - DNA, 75 bp, artificial
  S .delta. 1R-2F
gcgatggtggcggtggcgaagaatactccgcaatgcgcgaccagtacatgcgcaccggtgaaggtggcggtggca

SEQ ID NO: 18 - DNA, 75 bp, artificial
  S .delta. 1R-2R
acattgccaccgccaccttcaccggtgcgcatgtactggtcgcgcattgcggagtattcttcgccaccgccacca

SEQ ID NO: 19 - protein, 15 aa, artificial
  ras free epitope
EEYSAMRDQVMRTGE

SEQ ID NO: 20 - protein, 22 aa, Bacteriophage T4
GLLRLNGDYVQGSNNVQFYADG

SEQ ID NO: 21 - protein, 16 aa, artificial
  modified portion of Bacteriophage T4 gene 37
GLLRLNGDNVQFYADG

SEQ ID NO: 22 - protein, 21 aa, artificial
  modified portions of Bacteriophage T4 gene 37
GLLRLNGDGGGGGNVQFYADG

SEQ ID NO: 23 - protein, 38 aa, artificial
  portion of Bacteriophage T4 gene 37 plus ras
GLLRLNGDGGGGARGVGKSALTIQLIGGGGNVQFYADG

SEQ ID NO: 24 - protein, 39 aa, artificial
  portion of Bacteriophage T4 gene 37 plus ras
GLLRLNGDGGGGEEYSAMRDQYMRTGEGGGGNVQFYADG